(19) United States
(12) Patent Application Publication
(10) Pub. No.: US 2025/0268605 A1
(43) Pub. Date: Aug. 28, 2025

(54) METHODS, SYSTEMS, AND DEVICES FOR THE OCCLUSION OF THE LEFT ATRIAL APPENDAGE

(71) Applicant: APPLIED CARDIOVASCULAR SOLUTIONS, LLC, Austin, TX (US)

(72) Inventors: F. Javier OTERO, Austin, TX (US); Joseph J. BEAMAN, Austin, TX (US); Rodney Paul HORTON, Austin, TX (US); Andrea NATALE, Austin, TX (US); Joel W. BARLOW, Austin, TX (US)

(21) Appl. No.: 18/859,907

(22) PCT Filed: Apr. 25, 2023
(86) PCT No.: PCT/US2023/019797

§ 371 Date: Oct. 24, 2024

Related U.S. Application Data
(60) Provisional application No. 63/334,639, filed on Apr. 25, 2022.

(51) Int. Cl. A61B 17/12; A61B 17/00; A61L 24/00; A61L 24/04

(52) U.S. Cl. CPC A61B 17/12195; A61B 17/12122; A61B 17/12172; A61B 17/12177; A61L 24/0015; A61L 24/043; A61B 2017/00557; A61B 2017/00893; A61B 2017/00942; A61L 2430/36

(57) ABSTRACT

A method of occluding a left atrial appendage (LAA) of a patient comprising: injecting a photocurable biomaterial into the LAA of the patient; and irradiating the photocurable biomaterial with actinic radiation, thereby inducing crosslinking of the photocurable biomaterial in situ in the LAA to form an interpenetrating network (IPN) or semi-interpenetrating network (sIPN) comprising a first network polymer and a second network polymer, wherein the first network polymer comprises a hydrophilic polymer; and wherein the second network polymer comprises a silicone rubber.

### CROSS-REFERENCE TO RELATED APPLICATIONS

This application claims benefit of U.S. Provisional Application No. 63/334,639, filed Apr. 25, 2022, which is hereby incorporated herein by reference in its entirety.

### FIELD

The present disclosure is generally related to methods, systems, and devices for occluding the left atrial appendage (LAA) of a patient's heart.

### BACKGROUND

Embolic stroke is a leading cause of death and disability among adults. The most common cause of embolic stroke emanating from the heart is thrombus formation due to atrial fibrillation (AF). AF is an arrhythmia of the heart that results in a rapid and chaotic heartbeat, producing decreased cardiac output and leading to irregular and turbulent blood flow in the vascular system.

In the case of patients who exhibit AF and develop an atrial thrombus, clot formation typically occurs in the left atrial appendage (LAA) of the patient's heart. The LAA is a small cavity formed within the lateral wall of the left atrium between the mitral valve and the root of the left pulmonary vein. In normal hearts, the LAA contracts in conjunction with the rest of the left atrium during the cardiac cycle; however, in the case of patients suffering from AF, the LAA often fails to contract with any vigor. As a consequence, blood can stagnate within the LAA, resulting in thrombus formation.

Elimination or containment of thrombus formed within the LAA offers the potential to significantly reduce the incidence of stroke in patients suffering from AF. Pharmacological therapies, for example the oral or systemic administration of anticoagulants such as warfarin, are often used to prevent thrombus formation. However, anticoagulant therapy is often undesirable or unsuccessful due to medication side effects (e.g., hemorrhage), interactions with foods and other drugs, and lack of patient compliance.

Invasive surgical or thorascopic techniques have been used to obliterate the LAA, however, many patients with AF are not suitable candidates for such surgical procedures due to a compromised condition or having previously undergone cardiac surgery. In addition, the perceived risks of surgical procedures often outweigh the potential benefits.

Recently, percutaneous occlusion implants for use in the LAA have been investigated as alternatives to anticoagulant therapy. However, these implants are relatively non-conforming. Due to the non-uniform shape of the LAA, existing implants cannot completely seal the opening of the LAA in all patients. As a consequence, approximately 15% of patients receiving these implants experience incomplete LAA closure, necessitating prolonged treatment with anticoagulants. The anatomy of the left atrium and LAA of some patients also precludes the use of such implants. In addition, the occlusion implants can also cause life-threatening perforations of the LAA during the placement procedure.

More effective methods of occluding cavities or passageways in a patient, in particular cavities or passageways in the cardiovascular system of a patient, such as the LAA, offer the potential to improve patient outcomes while eliminating the undesirable consequences of existing therapies.

### SUMMARY

Provided are methods, systems, and devices for occluding the LAA of a patient's heart. These methods, systems, and devices can be used to decrease the rate of thromboembolic events associated with AF by occluding the LAA.

Methods for occluding the LAA of a patient can involve injecting a photocurable biomaterial into the LAA of the patient. The photocurable biomaterial is a flowable, fluid composition when injected, allowing it to comply with the irregular shape of the interior of the LAA. Prior to or even after injection into the LAA, the photocurable biomaterial can be irradiated with actinic radiation, thereby inducing crosslinking of the photocurable biomaterial in situ in the LAA to form an interpenetrating network (IPN) or semi-interpenetrating network (sIPN). The interpenetrating network (IPN) or semi-interpenetrating network (sIPN) can comprise a first network polymer and a second network polymer. The first network polymer can comprise, for example, a hydrophilic polymer. The second network polymer can comprise, for example, a silicone rubber (e.g., a hydrophilic silicone rubber).

The resulting interpenetrating network (IPN) or semi-interpenetrating network (sIPN) can be a solid material that fills and occupies the internal volume of the LAA. Because of the compliant nature of the fluid, photocurable biomaterial, the resulting interpenetrating network (IPN) or semi-interpenetrating network (sIPN) can be interpenetrated by trabeculae present in the LAA. In this way, the interpenetrating network (IPN) or semi-interpenetrating network (sIPN) can function as an occlusive body, filling and occupying the internal volume of the LAA without adversely impacting cardiac function. In some embodiments, the interpenetrating network (IPN) or semi-interpenetrating network (sIPN) can have a volume within at least 40% of the original internal volume of the patient's LAA prior to any surgical manipulation of the patient's LAA (e.g., prior to injection of the photocurable biomaterial.

The hydrophilic polymer can be selected from the group consisting of polyethers, polyacrylates, polyesters, polyanhydrides, polyols, polypeptides, polyvinyl alcohols, proteins, polysaccharides, gelatins, elastins, collagens, celluloses, methylcelluloses, hyaluronic acid, dextrans, alginates, copolymers thereof, and derivatives thereof.

In some embodiments, the hydrophilic polymer can comprise a non-biodegradable polymer. In some embodiments, the hydrophilic polymer can comprise a synthetic polymer.

In certain examples, the hydrophilic polymer can comprise a hydrophilic polyacrylate, such as poly(hydroxyethyl)methacrylate or a copolymer thereof.

In certain examples, the hydrophilic polymer can comprise a hydrophilic urethane acrylate.

In some embodiments, the photocurable biomaterial can comprise a bifunctional epoxy siloxane monomer. Optionally, the bifunctional epoxy siloxane monomer can comprise one or more hydrophilic functional groups, such as one or more hydroxy groups, one or more carboxylic acid groups, or a combination thereof.

In some embodiments, the photocurable biomaterial can further comprise one or more additional epoxy monomers, such as one or more polyfunctional epoxy siloxane monomers. The one or more polyfunctional epoxy siloxane monomers comprise at least three epoxy groups (e.g., three epoxy groups per monomer, four epoxy groups per monomer, five epoxy groups per monomer, or six epoxy groups per monomer). Optionally, the one or more additional epoxy monomers can comprise one or more hydrophilic functional groups, such as one or more hydroxy groups, one or more carboxylic acid groups, or a combination thereof.

When the photocurable biomaterial includes both a bifunctional epoxy siloxane monomer and one or more polyfunctional epoxy siloxane monomers, the one or more polyfunctional epoxy siloxane monomers and the bifunctional epoxy siloxane monomer can be present in the photocurable biomaterial at a molar ratio of from 0.01:100 to 15:100, such as from 0.05:100 to 10:100 or from 0.1:100 to 5:100.

In some embodiments, the photocurable biomaterial can further comprise one or more (meth)acrylate monomers.

In some embodiments, first network polymer and the second network polymer can be co-continuous.

In some embodiments, crosslinking of the photocurable biomaterial in situ in the LAA forms an interpenetrating network (IPN).

In some embodiments, the first network polymer comprises at least 30% by weight of the IPN or sIPN (e.g., from 30% by weight to 80% by weight), based on the total weight of all network polymers forming the IPN or sIPN.

In some embodiments, the photocurable biomaterial can have a viscosity of from 1 cP to 10,000 cP (e.g., 1 cP to 200 cP) at 25° C. Once crosslinked, the IPN or sIPN can exhibit a viscosity of at least 500,000 cP at body temperature (e.g., at 37° C.). Properly cross-linked, the biomaterial becomes essentially one molecule with infinite molecular weight and viscosity.

In some embodiments, irradiating the photocurable biomaterial with actinic radiation can comprise delivering at least 5 J/cm³ of energy to the photocurable biomaterial.

In some embodiments, methods can further involve positioning an occlusion device within the ostium of the LAA. The occlusion device can be positioned within the ostium of the LAA before photocrosslinking of the photocurable biomaterial. The occlusion device can comprise an occluder portion and an anchor portion coupled to the occluder portion. When the occlusion device is positioned within the ostium of the LAA, the anchor portion extends into the internal volume of the LAA.

Upon crosslinking, the resulting interpenetrating network (IPN) or semi-interpenetrating network (sIPN) fills and occupies the internal volume of the LAA. Because of the compliant nature of the photocurable biomaterial, the resulting interpenetrating network (IPN) or semi-interpenetrating network (sIPN) can be interpenetrated by both the anchor portion of the occlusion device and trabeculae present in the LAA. In this way, the interpenetrating network (IPN) or semi-interpenetrating network (sIPN) ensures that the occlusion device is retained within the ostium of the LAA. In addition, the occlusion device can isolate the interpenetrating network (IPN) or semi-interpenetrating network (sIPN) from blood present in the left atrium, provide a scaffold for endothelialization, of a combination thereof.

In some embodiments, the occlusion device can be positioned within the ostium of the LAA prior to injection of the photocurable biomaterial into the LAA. For example, methods for occluding the LAA of a patient can involve positioning an occlusion device within the ostium of the LAA, wherein the occlusion device comprises an occluder portion comprising a proximal end and a distal end, the proximal end coupled to a hub having an injection lumen passing axially therethrough; and an anchor portion operably coupled to the occluder portion. The occlusion device can be positioned within the ostium of the LAA such that the anchor portion extends into the internal volume of the LAA. A photocurable biomaterial can then be injected into the LAA of the patient through the injection lumen of the occlusion device. The photocurable biomaterial can be flowable and conform to the internal anatomy of the LAA prior to crosslinking. Subsequently, methods can involve irradiating the photocurable biomaterial with actinic radiation, thereby inducing crosslinking of the photocurable biomaterial in situ in the LAA to form the IPN or sIPN that fills and occupies the internal volume of the LAA. The occlusion device can be retained within the ostium of the LAA until irradiation and photocrosslinking have occurred and the photocurable biomaterial has solidified to form the IPN or sIPN.

In other embodiments, the occlusion device can be positioned within the ostium of the LAA after injection of the photocurable biomaterial into the LAA. For example, methods for occluding the LAA of a patient can involve injecting a photocurable biomaterial into the LAA of the patient. The photocurable biomaterial can be flowable, allowing it to comply with the irregular shape of the interior of the LAA. An occlusion device can then be positioned within the ostium of the LAA. The occlusion device can comprise an occluder portion and an anchor portion operably coupled to the occluder portion. When the occlusion device is positioned within the ostium of the LAA, the anchor portion extends into the internal volume of the LAA. The occlusion device can be retained within the ostium of the LAA while the photocurable biomaterial is irradiated with actinic radiation, thereby inducing crosslinking of the photocurable biomaterial in situ in the LAA to form the IPN or sIPN that fills and occupies the internal volume of the LAA. Because of the compliant nature of the photocurable biomaterial, the resulting IPN or sIPN can be interpenetrated by both the anchor portion of the occlusion device and trabeculae present in the LAA.

In other embodiments, the occlusion device can comprise an occluder portion comprising a proximal end and a distal end, the proximal end coupled to a hub having an injection lumen passing axially therethrough. Methods of occluding the LAA of a patient can comprise positioning the occlusion device within the ostium of the LAA, injecting a photocurable biomaterial into the LAA of the patient through the injection lumen. The photocurable biomaterial can then be irradiated with actinic radiation, thereby inducing crosslinking of the photocurable biomaterial in situ in the LAA to form the IPN or sIPN that fills and occupies the internal volume of the LAA. An anchoring portion can then be advanced through the injection lumen and coupling the anchoring portion to the occlusion device, wherein when the anchoring portion is coupled to the occlusion device, the anchor portion extends into the internal volume of the LAA. The anchor portion can be structured to be advanced through the IPN or sIPN (e.g., the anchor portion can comprise one or more structures similar to barbed needles).

In some methods, the delivery catheter can comprise an element configured to irradiate the photocurable biomaterial with actinic radiation, such as a water light pipe, light source (e.g., UV LED), or a combination thereof.

In some embodiments, the photocurable biomaterial can further comprise a silencing agent dissolved or dispersed therein. In these embodiments, the resulting IPN or sIPN formed in situ in the LAA upon crosslinking can comprise a silencing agent. The silencing agent can comprise any suitable agent (small molecule or biologic) that can be locally released from the photocurable biomaterial (and/or the biocompatible polymer matrix) and eliminates contractility of cardiac tissue in the walls of the LAA (e.g., by interrupting electrical signals, inducing apoptosis, etc.). In some embodiments, the IPN or sIPN can provide for localized, controlled release of the silencing agent to cardiac tissue in the walls of the LAA over a period of at least 2 weeks. In certain embodiments, the apoptotic agent can comprise aclarubicin, an apoptosis gene modulator, an apoptosis regulator, an arginine deaminase, clotrimazole, curacin A, etoposide, gemcitabine, a ras inhibitor, a ras-GAP inhibitor, a topoisomerase inhibitor such as topotecan or camptothecin, a taxane such as docetaxel or paclitaxel, an anthracycline, a cyclophosphamide, a vinca alkaloid, a platinum-based chemotherapeutic agent such as cisplatin or carboplatin, 5-fluoro-uracil, gemcitabine, capecitabin, navelbine, zoledronate, venetoclax, ABT-737, or any combination thereof.

### BRIEF DESCRIPTION OF THE FIGURES

FIG. 1 is an anterior illustration of a heart, including proximal portions of the great vessels.

FIG. 2A is a perspective view of an example occlusion device and a distal portion of an example delivery system. FIG. 2B is a partial cross-sectional view of the occlusion device, taken along section line IA of FIG. 2A. FIG. 2C is an enlarged section view of an occluder portion, taken from detail 2B of FIG. 2B.

FIG. 3A is a side view of an example occlusion device and an example delivery system FIG. 3B is a side view of an example occlusion device employed with the delivery system of FIG. 3B, depicting the occlusion device being implanted in a left atrial appendage.

FIG. 4 is a perspective view of the occlusion device of FIG. 3A, depicting the medical device in a fully expanded position.

FIG. 5 is a side view of the occlusion device shown in FIG. 4.

FIG. 6A is a side view of an example occlusion device and an example delivery system

FIG. 6B is a side view of an example occlusion device employed with the delivery system of FIG. 6B, depicting the occlusion device being implanted in a left atrial appendage.

FIGS. 7A-7C illustrate another example occlusion device.

FIG. 8A is a schematic illustration of an exemplary first catheter body and first balloon, as described herein. As shown, the first catheter body can have a proximal end portion, a distal end portion having a tip, and a wall that circumferentially encloses a primary opening. FIG. 8B is a cross-sectional side view of the first catheter body taken along line 1B-1B of FIG. 8A. As shown, the first catheter body can have at least one inflation channel within the wall of the first catheter body. The primary opening of the first catheter body can extend an entire length of the first catheter body. FIG. 8C is a close-up view of a portion of the first catheter body of FIG. 8A showing at least one outlet opening defined therein to provide fluid communication between the at least one inflation channel and the interior space of the first balloon, as described herein.

FIG. 8D is a cross-sectional side view of the portion of the first catheter body of FIG. 8C taken along line 1D-1D, as described herein.

FIG. 9A is a schematic illustration of an exemplary second catheter body and second balloon, as described herein. As shown, the second catheter body can have a proximal end portion, a distal end portion having a tip, and a wall that circumferentially encloses a primary opening. FIG. 9B is a cross-sectional side view of the second catheter body taken along line 2B-2B of FIG. 9A, as described herein. As shown, the second catheter body can have at least one inflation channel within the wall of the second catheter body, and the primary opening of the second catheter body can extend an entire length of the second catheter body. FIG. 9C is a close-up view of a portion of the second catheter body of FIG. 9A showing at least one outlet opening defined therein to provide fluid communication between the at least one inflation channel and the interior space of the second balloon, as described herein.

FIG. 10A is a schematic illustration of an exemplary third catheter body, as described herein. As shown, the third catheter body can include a proximal end portion, a distal end portion, and a wall structure that defines at least one injection channel extending from the proximal end portion toward the distal end portion. FIG. 10B is a cross-sectional side view of the third catheter body taken along line 3B-3B of FIG. 10A, which shows the least one injection channel. FIG. 10C is a close-up view of the distal end portion of the third catheter body of FIG. 10A, as described herein. FIG. 10D is a cross-sectional side view of the distal end portion taken along line 3D-3D of FIG. 10C. As shown and described herein, the distal end portion can at least one outlet opening positioned in fluid communication with the at least one injection channel.

FIG. 11 is a perspective view of an exemplary catheter assembly, as described herein.

FIGS. 12A-12C illustrate an example method of occluding the LAA.

FIGS. 13A-13D illustrate an example method of occluding the LAA.

FIGS. 14A-14E illustrate an example method of occluding the LAA.

### DETAILED DESCRIPTION

Before the present methods, compositions, systems, articles, devices, and/or methods are disclosed and described, it is to be understood that the aspects described below are not limited to specific compounds, specific devices, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Examples of longer chain alkyl groups include, but are not limited to, a palmitate group. A “lower alkyl” group is an alkyl group containing from one to six carbon atoms.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulfur, or phosphorus.

The term “aryl group” as used herein is any carbon-based aromatic group including, but not limited to, benzene, naphthalene, etc. The term “aryl group” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. In one aspect, the heteroaryl group is imidazole. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “nucleophilic group” includes any groups capable of reacting with an activated ester. Examples include amino groups, thiols groups, hydroxyl groups, and their corresponding anions.

The term “carboxyl group” includes a carboxylic acid and the corresponding salt thereof.

The term “amino group” as used herein is represented as the formula —NHRR′, where R and R′ can be any organic group including alkyl, aryl, carbonyl, heterocycloalkyl, and the like, where R and R′ can be separate groups or be part of a ring. For example, pyridine is a heteroaryl group where R and R′ are part of the aromatic ring.

The term “treat” as used herein is defined as maintaining or reducing the symptoms of a pre-existing condition. The term “prevent” as used herein is defined as eliminating or reducing the likelihood of the occurrence of one or more symptoms of a disease or disorder. The term “reduce” as used herein is the ability of the in situ solidifying complex coacervate described herein to completely eliminate the activity or reduce the activity when compared to the same activity in the absence of the complex coacervate.

“Subject” refers to mammals including, but not limited to, humans, non-human primates, sheep, dogs, rodents (e.g., mouse, rat, etc.), guinea pigs, cats, rabbits, cows, and non-mammals including chickens, amphibians, and reptiles.

“Physiological conditions” refers to condition such as pH, temperature, etc. within the subject. For example, the physiological pH and temperature of a human is 7.2 and 37° C., respectively.

The term “interpenetrating polymer network” (IPN) refers to a polymer network that comprises an intermixture of two or more polymers that are physically entangled but not chemically linked (i.e., two or more networks which are at least partially interlaced on a polymer scale but not covalently bonded to each other). Generally, the polymers in the IPN cannot be separated unless chemical bonds are broken. The two or more networks can be envisioned to be entangled in such a way that they are concatenated and cannot be pulled apart, but not bonded to each other by any chemical bond. The term IPN includes networks formed by simultaneous synthesis processes, networks formed by sequential synthesis processes, and “semi-IPN” systems that include a linear, non-crosslinked polymer that is physically entangled within a crosslinked polymer network. The term IPN can also include interconnected polymer networks that include a limited amount of inter-network chemical links.

The term “biocompatible”, as used herein, refers to having the property of being biologically compatible by not producing a toxic, injurious, or immunological response in living tissue.

The phrase “polydispersity index” refers to the ratio of the “weight average molecular weight” to the “number average molecular weight” for a particular polymer; it reflects the distribution of individual molecular weights in a polymer sample.

The phrase “weight average molecular weight” refers to a particular measure of the molecular weight of a polymer. The weight average molecular weight is calculated as follows: determine the molecular weight of a number of polymer molecules; add the squares of these weights; and then divide by the total weight of the molecules.

The phrase “number average molecular weight” refers to a particular measure of the molecular weight of a polymer. The number average molecular weight is the common average of the molecular weights of the individual polymer molecules. It is determined by measuring the molecular weight of n polymer molecules, summing the weights, and dividing by n.

“Biodegradable” means that a material is capable of being broken down physically and/or chemically within cells or within the body of a subject, e.g., by hydrolysis under physiological conditions and/or by natural biological processes such as the action of enzymes present within cells or within the body, and/or by processes such as dissolution, dispersion, etc., to form smaller chemical species which can typically be metabolized and, optionally, used by the body, and/or excreted or otherwise disposed of. For purposes of the present invention, a polymer or hydrogel whose molecular weight decreases over time in vivo due to a reduction in the number of monomers is considered biodegradable. In certain embodiments, the hydrogel useful in vocal cord repair is not substantially biodegradable.

The term “crosslinked” as used herein describes a polymer with at least one covalent bond that is not found in the repeating units of the polymer or found between repeating units of the polymer. The crosslinking bonds are typically between individual strands or molecules of the polymer; however, intramolecular crosslinking to form macrocyclic structures may also occur. The crosslinks are formed between any two functional groups of the polymer (e.g., at the ends, on the side chains, etc.). In certain embodiments, the crosslinks are formed between terminal acrylate units of the polymers. Also, any type of covalent bond may form the crosslink (e.g., carbon-carbon, carbon-oxygen, carbon-nitrogen, oxygen-nitrogen, sulfur-sulfur, oxygen-phosphorus, nitrogen-nitrogen, oxygen-oxygen, etc.). The resulting crosslinked material may be branched, linear, dendritic, etc. In certain embodiments, the crosslinks form a 3-D network of crosslinks. The crosslinks may be formed by any chemical reaction that results in the covalent bonds. Typically, the crosslinks are created by free radical initiated reactions, for example, with a photoinitiator or thermal initiator.

“Viscosity” refers to a measurement of the resistance to flow of a liquid at a given temperature. Viscosity may be determined using a variety of methods and instruments known in the art. For example, a polymer is first weighed and then dissolved in an appropriate solvent. The solution and viscometer are placed in a constant temperature water bath. Thermal equilibrium is obtained within the solution. The liquid is then brought above the upper graduation mark on the viscometer. The time for the solution to flow from the upper to lower graduation marks is recorded. Viscosity of a solution comprising a polymer may be determined in accordance with ASTM Book of Standards, Practice for Dilute Solution Viscosity of Polymers (ASTM D2857), Volume 08.01, June 2005 or relevant ASTM standards for specific polymers. Solubility may be tested at a temperature of between 20 and 40° C., e.g., approximately 25-37° C., e.g., approximately 37° C., or any intervening value of the foregoing ranges. For example, solubility may be determined at approximately pH 7.0-7.4 and approximately 37° C.

“Elastic shear modulus” of a material is a mathematical description of a material's tendency to be deformed elastically (i.e., non-permanently) when a force is applied parallel to one of its surfaces while its opposite face experiences an opposing force (e.g., friction). Elastic shear modulus is calculated as the ratio of shear stress to shear strain. For example, if a force of 1 N is applied tangentially (on the xy plane) to a surface of an area of 1 m² and produces a change in the shape by 1% (strain=0.0l) in the xy plane, then the elastic shear modulus of the material is 1/0.01=100 Pa.

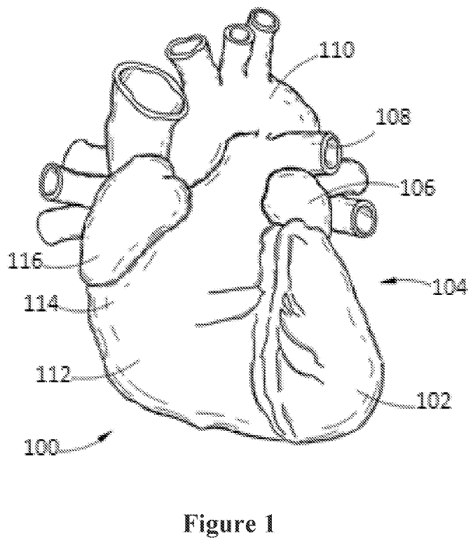

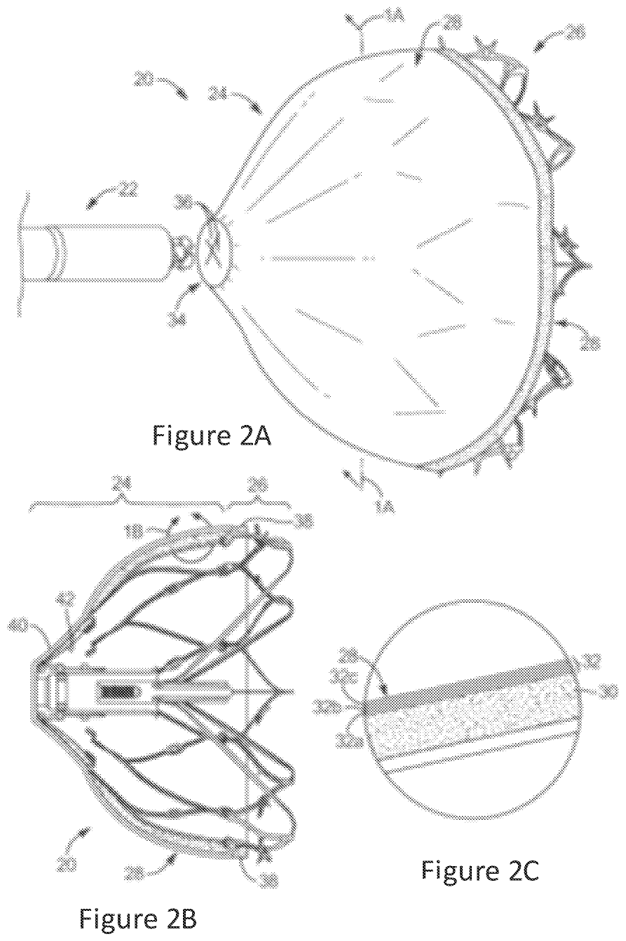

To facilitate understanding of the physiology associated with the methods, compositions, and devices described herein, FIG. 1 illustrates the anatomy of the human heart (100). Referring to FIG. 1, the heart 100 is illustrated to show certain portions including the left ventricle (102), the left atrium (104), the LAA (106), the pulmonary artery (108), the aorta (110), the right ventricle (112), the right atrium (114), and the right atrial appendage (116). The left atrium is located above the left ventricle, and is separated from the left ventricle by the mitral valve (not illustrated). As shown in FIG. 2A, the LAA (106) can have an irregular finger-like or windsock shape with an opening (also referred to as an ostium, 120) approximately 1.5 cm in diameter. The internal volume of a normal LAA is approximately 9.3±3.5 mL. The LAA is normally in fluid communication with the left atrium such that blood flows in and out of the LAA as the heart beats.

Methods for occluding the LAA of a patient can involve injecting a photocurable biomaterial into the LAA of the patient. The photocurable biomaterial is a flowable, fluid composition when injected, allowing it to comply with the irregular shape of the interior of the LAA. The photocurable biomaterial can be irradiated with actinic radiation, immediately prior to injection or after injection into the LAA thereby inducing crosslinking of the photocurable biomaterial in situ in the LAA to form an interpenetrating network (IPN) or semi-interpenetrating network (sIPN). The interpenetrating network (IPN) or semi-interpenetrating network (sIPN) can comprise a first network polymer and a second network polymer. The first network polymer can comprise, for example, a hydrophilic polymer. The second network polymer can comprise, for example, a silicone rubber (e.g., a hydrophilic silicone rubber, or a hydrophobic).

The resulting interpenetrating network (IPN) or semi-interpenetrating network (sIPN) can be a solid material that fills and occupies the internal volume of the LAA. Because of the compliant nature of the fluid, photocurable biomaterial, the resulting interpenetrating network (IPN) or semi-interpenetrating network (sIPN) can be interpenetrated by trabeculae present in the LAA. In this way, the interpenetrating network (IPN) or semi-interpenetrating network (sIPN) can function as an occlusive body, filling and occupying the internal volume of the LAA without adversely impacting cardiac function. In some embodiments, the interpenetrating network (IPN) or semi-interpenetrating network (sIPN) can have a volume within at least 40% (e.g., at least 50%, at least 60%, at least 70%, at least 75%, at least 80%, or at least 90%) of the original internal volume of the patient's LAA prior to any surgical manipulation of the patient's LAA (e.g., prior to injection of the photocurable biomaterial.

### Photocurable Biomaterials

The methods described herein involve injection of a photocurable biomaterial into the LAA of a subject. Once introduced into the LAA, the photocurable biomaterial can be irradiated with actinic radiation, immediately prior to injection or after injection, thereby inducing crosslinking of the photocurable biomaterial in situ in the LAA to form an interpenetrating network (IPN) or semi-interpenetrating network (sIPN). The interpenetrating network (IPN) or semi-interpenetrating network (sIPN) can comprise a first network polymer and a second network polymer. The first network polymer can comprise, for example, a hydrophilic polymer. The second network polymer can comprise, for example, a silicone rubber (e.g., a hydrophilic or hydrophobic silicone rubber—either can be used provided that at least one of the polymers is hydrophilic). The resulting interpenetrating network (IPN) or semi-interpenetrating network (sIPN) can be a solid material that fills and occupies the internal volume of the LAA.

The photocurable biomaterial as well as the resultant IPN or sIPN can be selected to possess suitable materials properties (e.g., viscosity, cohesive strength, adhesive strength, elasticity, degradation rate, swelling behavior, cure time, etc.) for use in occlusion of the LAA.

For example, the IPN or sIPN can exhibit an equilibrium swelling ratio suitable for occlusion of the LAA. Swelling refers to the uptake of water or biological fluids by the IPN or sIPN. The swelling of the IPN or sIPN can be quantified using the equilibrium swelling ratio, defined as the mass of the IPN or sIPN at equilibrium swelling (i.e., the materials maximum swollen weight) divided by the mass of the IPN or sIPN prior to swelling (e.g., immediately following crosslinking). In many cases, equilibrium swelling is reached within a relatively short period of time (e.g., within about 24-48 hours).

In some embodiments, the IPN or sIPN exhibits an equilibrium swelling ratio of less than about 10 (e.g., less than about 9.5, less than about 9.0, less than about 8.5, less than about 8.0, less than about 7.5, less than about 7.0, less than about 6.5, less than about 6.0, less than about 5.5, less than about 5.0, less than about 4.5, less than about 4.0, less than about 3.5, less than about 3.0, less than about 2.5, less than about 2.0, less than about 1.5, less than about 1.0, or less than about 0.5). In some embodiments, the IPN or sIPN exhibits an equilibrium swelling ratio of greater than 0 (e.g., greater than about 0.5, greater than about 1.0, greater than about 1.5, greater than about 2.0, greater than about 2.5, greater than about 3.0, greater than about 3.5, greater than about 4.0, greater than about 4.5, greater than about 5.0, greater than about 5.5, greater than about 6.0, greater than about 6.5, greater than about 7.0, greater than about 7.5, greater than about 8.0, or greater than about 8.5, greater than about 9.0, or greater than about 9.5).

The IPN or sIPN can exhibit an equilibrium swelling ratio ranging from any of the minimum values described above to any of the maximum values described above. For example, the IPN or sIPN can exhibit an equilibrium swelling ratio of from greater than 0 to about 10.0 (e.g., from about 2.0 to about 8.0, of from about 2.5 to about 6.0). In certain embodiments, the the IPN or sIPN can exhibit an equilibrium swelling ratio of less than about 1.5.

In some embodiments, the IPN or sIPN exhibits a volumetric swelling ratio of less than about 15 (e.g., less than about 12, less than about 10, less than about 9, less than about 8, less than about 7, less than about 6, less than about 5, less than about 4, less than about 3, less than about 2, or less than about 1). In some embodiments, the IPN or sIPN exhibits an equilibrium swelling ratio of greater than 0 (e.g., greater than about 1, greater than about 2, greater than about 3, greater than about 4, greater than about 5, greater than about 6, greater than about 7, greater than about 8, greater than about 9, greater than about 10, or greater than about 12).

The IPN or sIPN can exhibit a volumetric swelling ratio ranging from any of the minimum values described above to any of the maximum values described above. For example, the IPN or sIPN can exhibit a volumetric swelling ratio of from greater than 0 to about 15 (e.g., from about 2 to about 15). In certain embodiments, the the IPN or sIPN can exhibit a volumetric swelling ratio of less than about 2.

The IPN or sIPN can have mechanical properties that are compatible with cardiac function, such that the presence of the IPN or sIPN within the LAA does not substantially impede or inhibit cardiac function. For example, the IPN or sIPN can be formed to be at least partially compliant with the constrictive action of the heart muscle throughout the cardiac cycle. Suitable IPN or sIPN can have elastic moduli ranging from about 100 to about 700 kPa. In certain examples, the IPN or sIPN can have a Shore A moduli of less than 20-25.

(In some cases, IPN or sIPN is formed to have an elastic modulus similar to that of cardiac tissue. In some embodiments, the IPN or sIPN has an elastic modulus greater than about 5 kPa (e.g., greater than about 6 kPa, greater than about 7 kPa, greater than about 8 kPa, greater than about 9 kPa, greater than about 10 kPa, greater than about 11 kPa, greater than about 12 kPa, greater than about 13 kPa, greater than about 14 kPa, greater than about 15 kPa, greater than about 16 kPa, greater than about 17 kPa, greater than about 18 kPa, or greater than about 19 kPa. In some embodiments, the IPN or sIPN has an elastic modulus of less than about 20 kPa (e.g., less than about 19 kPa, less than about 18 kPa, less than about 17 kPa, less than about 16 kPa, less than about 15 kPa, less than about 14 kPa, less than about 13 kPa, less than about 12 kPa, less than about 11 kPa, less than about 10 kPa, less than about 9 kPa, less than about 8 kPa, less than about 7 kPa, or less than about 6 kPa).

The IPN or sIPN can have an elastic modulus ranging from any of the minimum values described above to any of the maximum values described above. For example, the IPN or sIPN can have an elastic modulus of from about 5 kPa to about 20 kPa (e.g., from about 9 kPa to about 17 kPa, from about 10 kPa to about 15 kPa, or from about 8 kPa to about 12 kPa).

In other embodiments, the IPN or sIPN can have an elastic modulus of greater than 20 kPa (e.g., from greater than 20 kPa to about 13000 kPa, from greater than 20 kPa to 10000 kPa).

The IPN or sIPN can have a cohesive strength suitable for occlusion of the LAA. Cohesive strength (also referred to as burst strength) refers to the ability of the IPN or sIPN to remain intact (i.e., not rupture, tear or crack) when subjected to physical stresses or environmental conditions. The cohesive strength of the IPN or sIPN can be measured using methods known in the art, for example, using the standard methods described in ASTM F-2392-04 (standard test for the burst strength of surgical sealants). In some embodiments, the IPN or sIPN has a cohesive strength effective such that the IPN or sIPN remains intact (e.g., does not fragment or break apart into smaller pieces which exit the LAA) for at least 90 days.

When properly cross-linked and reacted, the biomaterial should be one molecule with infinite viscosity. Immediately after injection, prior to significant chemical reaction, the reaction mixture can have a viscosity which minimizes migration of IPN or sIPN out of the LAA. Simultaneously, the reaction mixture needs sufficiently low viscosity to permit injection through small, typically 8.5 Fr catheters. This constraint limits the viscosity in the catheter to 600-1000 cP. Higher reaction mixture viscosity in the LAA after injection and prior to reaction can be achieved by the addition of appropriate shear-thinning thixotropic materials to the reaction mixture. Said thixotropes can be constructed from the same materials used in the IPN or sIPN by controlling its architecture. Alternately, appropriate biocompatible inorganic materials (e.g. finely divided calcium hydroxy apatite) can also act as thixotropes by forming ionic networks within the reaction mixture. In some embodiments, the IPN or sIPN has a viscosity of at least 50,000 cP (e.g., at least 60,000 cP, at least 70,000 cP, at least 75,000 cP, at least 80,000 cP, at least 90,000 cP, at least 100,000 cP, at least 250,000 cP, or more) at body temperature (e.g., at 37° C.).

The IPN or sIPN can also be selected such that is it retained at the site of occlusion (e.g., inside the LAA) by a combination of adhesion to the tissues at the site of occlusion and mechanical interaction with the anatomy at the site of occlusion. For example, the IPN or sIPN can have an adhesive strength suitable for occlusion of the LAA. Adhesive strength refers to the ability of the IPN or sIPN to remain attached to the tissues at the site of administration (e.g., the interior of the LAA) when subjected to physical stresses or environmental conditions. In some embodiments, the IPN or sIPN has an adhesive strength effective such that the IPN or sIPN within the LAA (e.g., does not exit the LAA) for at least 90 days. Mechanical forces, governed by a combination of properties which can include the swelling of the IPN or sIPN, the local anatomy at the site of injection (e.g., the particular 3-dimensional shape of the LAA and/or the surface texture of the LAA interior, and/or the presence of trabeculae within the patient's LAA), and the friction of the IPN or sIPN against tissue at the site of injection, can also contribute to retention of the IPN or sIPN at the site of injection.

The IPN or sIPN can be formed from materials which support (i.e., do not inhibit) endothelialization. Endothelialization refers to the growth and/or proliferation of endothelial cells on a surface, such as the blood-contacting surface, of the IPN or sIPN or a surface of the occlusion device. These materials can be biodegradable or non-biodegradable. A biodegradable material is one which decomposes under normal in vivo physiological conditions into components which can be metabolized or excreted.

In certain cases, the IPN or sIPN can be non-biodegradable. In some embodiments, the IPN or sIPN has a degradation rate such that about 25% or less by weight of the IPN or sIPN degrades within 90 days of curing, as measured using the standard method described in Example 1 (e.g., about 20% or less by weight, about 15% or less by weight, about 10% or less by weight, about 5% or less by weight, about 2.5% or less by weight, or less).

Suitable IPNs or sIPNs can be formed from a variety of natural and/or synthetic materials. In certain embodiments, the interpenetrating network (IPN) or semi-interpenetrating network (sIPN) can comprise a first network polymer and a second network polymer. The first network polymer can comprise, for example, a hydrophilic polymer. The second network polymer can comprise, for example, a silicone rubber (e.g., a hydrophilic or hydrophobic silicone rubber).

The photocurable biomaterial can be designed to rapidly cure in situ upon irradiation with actinic radiation. In some embodiments, the photocurable biomaterial has a cure time following irradiation, as measured using the standard method described in Example 1, of less than about 20 minutes (e.g., less than about 15 minutes, less than about 10 minutes, less than about 5 minutes, less than about 3 minutes, or less than about 1 minute). In some embodiments, irradiating the photocurable biomaterial with actinic radiation comprises delivering at least 5 J/cm³ of energy to the photocurable biomaterial.

The photocurable biomaterial injected into the LAA can have a low viscosity relative to the IPN or sIPN. This can allow the photocurable biomaterial to be readily injected, for example, via a hand-powered delivery device such as a syringe. This can provide a physician with a large degree of control over the flow rate of the photocurable biomaterial during injection, and allow the flow to be altered or stopped, as required, during the course of injection. The relatively low viscosity of the photocurable biomaterial relative to the IPN or sIPN also can allow the photocurable biomaterial to conform to the shape of the LAA prior to photocrosslinking and intimately interact with both trabeculae in the LAA as well as the anchor portion of the occlusion device (when used).

For example, in some embodiments, the photocurable biomaterial injected into the LAA has a viscosity of about 2,000 cP or less (e.g., about 1,500 cP or less, about 1,250 cP or less, about 1,000 cP or less, about 900 cP or less, about 800 cP or less, about 750 cP or less, about 700 cP or less, about 600 cP or less, about 500 cP or less, about 400 cP or less, about 300 cP or less, about 250 cP or less, about 200 cP or less, about 150 cP or less, about 100 cP or less, or. about 50 cP or less) at room temperature. In some embodiments, the viscosity of the photocurable biomaterial can be greater than the viscosity of human blood. In certain cases, the photocurable biomaterial is injected into the LAA has a viscosity of at least 1 cP (e.g., at least 2 cP, at least 2.5 cP, at least 5 cP, or at least 10 cP) at room temperature. The photocurable biomaterial can have a viscosity ranging from any of the minimum values described above to any of the maximum values described above.

Examples of suitable photocurable biomaterials are discussed in more detail below.

IPNs and sIPNs

IPNs can combine aspects of the characteristics of component chain polymer networks in ways that are distinct from those obtained by copolymerization or polymer blending. Unlike polymer blends, in which highly immiscible polymers can undergo extensive phase separation, the incompatible polymers in the IPN cannot phase separate. IPN formation permits polymers with very different properties (for example, a hydrophilic polymer and a hydrophobic polymer), to be combined to form mechanically robust hydrogels and elastomers. Examples of IPNs which can possess suitable biocompatibility and biomaterials properties for use in conjunction with the methods described herein include porous cross-linked polymer structures can be generated within a semi-IPN by selective solvent extraction of the linear component. The hydrophilicity and associated biocompatibility of a cross-linked network of silicone rubber can be improved (without adversely affecting the mechanical properties of the rubber) by swelling the rubber with 2-hydroxy ethyl methacrylate (HEMA) and polymerizing it to form a sequential IPN on the rubber surface. While this is not practical for an injectable system such as described herein, the copolymerization of silicone rubber oligomers and HEMA to form an IPN is possible and desirable. Materials formed from a porous poly(caprolactone) framework and a 3D-printed hydrogel cross-linked with UV light have also been prepared in which the porous network mechanically reinforces the reinforces the hydrogel and the hydrogel provides the hydrophilicity for biocompatibility.

In some embodiments, the photocurable biomaterial can comprise a composition that forms an IPN in situ by simultaneous polymerization and/or cross-linking (e.g., photopolymerization and/or photocrosslinking). For example, one-pot injectable mixtures of aliphatic epoxy and HEMA monomers can be polymerized (e.g., by light-initiated polymerization) to form parallel simultaneous polymerization routes (cationic for the epoxy and free radical for the methacrylate) with possibly some chain transfer between the epoxy and hydroxy moieties to form IPNs or sIPNs.

The interpenetrating network (IPN) or semi-interpenetrating network (sIPN) can comprise a first network polymer and a second network polymer. The first network polymer can comprise, for example, a hydrophilic polymer. The second network polymer can comprise, for example, a silicone rubber (e.g., a hydrophilic or hydrophobic silicone rubber).

In some embodiments, the hydrophilic polymer can be selected from the group consisting of polyethers, polyacrylates, polyesters, polyanhydrides, polyols, polypeptides, polyvinyl alcohols, proteins, polysaccharides, gelatins, elastins, collagens, celluloses, methylcelluloses, hyaluronic acid, dextrans, alginates, copolymers thereof, and derivatives thereof.

In some embodiments, the hydrophilic polymer can comprise a non-biodegradable polymer. In some embodiments, the hydrophilic polymer can comprise a synthetic polymer.

In certain examples, the hydrophilic polymer can comprise a hydrophilic polyacrylate, such as poly(hydroxyethyl)methacrylate or a copolymer thereof.

In certain examples, the hydrophilic polymer can comprise a hydrophilic urethane acrylate.

In some embodiments, the photocurable biomaterial can comprise a bifunctional epoxy siloxane monomer. Optionally, the bifunctional epoxy siloxane monomer can comprise one or more hydrophilic functional groups, such as one or more hydroxy groups, one or more carboxylic acid groups, or a combination thereof.

In some embodiments, the photocurable biomaterial can further comprise one or more additional epoxy monomers, such as one or more polyfunctional epoxy siloxane monomers. The one or more polyfunctional epoxy siloxane monomers comprise at least three epoxy groups (e.g., three epoxy groups per monomer, four epoxy groups per monomer, five epoxy groups per monomer, or six epoxy groups per monomer). Optionally, the one or more additional epoxy monomers can comprise one or more hydrophilic functional groups, such as one or more hydroxy groups, one or more carboxylic acid groups, or a combination thereof.

When the photocurable biomaterial includes both a bifunctional epoxy siloxane monomer and one or more polyfunctional epoxy siloxane monomers, the one or more polyfunctional epoxy siloxane monomers and the bifunctional epoxy siloxane monomer can be present in the photocurable biomaterial at a molar ratio of from 0.01:100 to 15:100, such as from 0.05:100 to 10:100 or from 0.1:100 to 5:100.

In some embodiments, the photocurable biomaterial can further comprise one or more (meth)acrylate monomers.

In some embodiments, first network polymer and the second network polymer can be co-continuous.

In some embodiments, crosslinking of the photocurable biomaterial in situ in the LAA forms an interpenetrating network (IPN).

In some embodiments, the first network polymer comprises at least 30% by weight of the IPN or sIPN (e.g., from 30% by weight to 80% by weight), based on the total weight of all network polymers forming the IPN or sIPN.

In some embodiments, the photocurable biomaterial can have a viscosity of from 1 cP to 10,000 cP at 25° C. Once crosslinked, the IPN or sIPN can exhibit a viscosity of at least 500,000 at body temperature (e.g., at 37° C.).

### Other Components

The photocurable biomaterials described above can further contain organic and/or inorganic additives, such as thixotropic agents, photoinitiator(s), stabilizers for stabilization of the precursor molecules in order to avoid premature crosslinking, and/or fillers which can result in an increase or improvement in the mechanical properties (e.g., cohesive strength and/or elastic modulus) of the resultant biocompatible matrix. Examples of stabilizing agents include radical scavengers, such as butylated hydroxytoluene or dithiothreitol.

In some embodiments, a bioactive agent can be incorporated into the photocurable biomaterial (and thus into the resultant IPN or sIPN). The bioactive agent can be a therapeutic agent, prophylactic agent, diagnostic agent, or combinations thereof. In some cases, the photocurable biomaterial (and thus the resultant IPN or sIPN) comprises an agent that promotes infiltration of cells onto or into the IPN or sIPN. For example, the agent can be an agent that promotes endothelialization. Promoting endothelialization refers to promoting, enhancing, facilitating, or otherwise increasing the attachment of, and growth of, endothelial cells on a surface of the IPN or sIPN. Examples of suitable agents that promote endothelialization are known in the art, and include growth factors (e.g., VEGF, PDGF, FGF, PlGF and combinations thereof), extracellular matrix proteins (e.g., collagen), and fibrin. In some cases, the photocurable biomaterial (and thus the resultant IPN or sIPN) comprises an anticoagulant, such as warfarin or heparin. In these cases, the anticoagulant can be locally delivered by elution from the resultant IPN or sIPN. In some cases, the photocurable biomaterial (and thus the resultant IPN or sIPN) comprises a contrast agent, such as gold, platinum, tantalum, bismuth, or combinations thereof to facilitate imaging of the photocurable biomaterial (e.g., during injection) or the resultant IPN or sIPN (e.g., to confirm complete occlusion of the LAA or monitor degradation of the IPN or sIPN).

In certain embodiments, the photocurable biomaterial (and thus the resultant IPN or sIPN) comprises a silencing agent. The silencing agent can comprise any suitable agent (small molecule or biological) that can be locally released from the photocurable biomaterial (and/or the IPN or sIPN) and eliminates contractility of cardiac tissue in the walls of the LAA (e.g., by interrupting electrical signals, inducing apoptosis, etc.). The IPN or sIPN can provide for localized, controlled release of the silencing agent to cardiac tissue in the walls of the LAA. For example, the IPN or sIPN can provide for localized release of an effective amount of the silencing agent to eliminate contractility of cardiac tissue in the walls of the LAA over a period of at least 24 hours, at least 48 hours, at least 1 week, at least 2 weeks, at least 1 month, at least 2 months, at least 3 months, or longer.

In some embodiments, the silencing agent can comprise an apoptotic agent. The term “apoptotic agent” as used herein is defined as a drug, toxin, compound, composition, or biological entity which bestows and/or activates apoptosis, or programmed cell death, onto a cell. Examples of apoptotic agents include aclarubicin, apoptosis gene modulators, apoptosis regulators, arginine deaminase, clotrimazole, curacin A, etoposide, gemcitabine, ras inhibitors, ras-GAP inhibitor, and topotecan. Other known apoptotic agents include taxanes including docetaxel and paclitaxel, anthracyclines, cyclophosphamide, vinca alkaloids, cisplatin, carboplatin, 5-fluoro-uracil, gemcitabine, capecitabin, navelbine, zoledronate, venetoclax, and ABT-737.

### Occlusion Devices

The occlusion device can be any device sized to be positioned within the ostium of the LAA, and which includes at least an occluder portion.

In some embodiments, the occlusion device can further include an anchor portion operably coupled to the occluder portion. In these embodiments, the occlusion device is further structured such that when the occlusion device is positioned within the ostium of the LAA, the anchor portion extends into the internal volume of the LAA.

In some embodiments, the occlusion device further comprises a hub. In such embodiments, the occluder portion can comprise a proximal end and a distal end, the proximal end coupled to the hub. Optionally, the anchor portion can be coupled to the occluder portion by way of the hub.

The occluder portion can be configured to move between an occluder-deployed state (e.g., where the occluder portion has a cross-sectional dimension effective to occlude the ostium of the LAA) and an occluder-nondeployed state (e.g., where the occluder portion has a cross-sectional dimension effective to allow the occlusion device to be advanced through a suitable delivery catheter). Likewise, the anchor portion can be configured to be moved between an anchor-deployed state (e.g., where the anchor portion extends into the internal volume of the LAA) and an anchor-nondeployed state (e.g., where the anchor portion has a cross-sectional dimension effective to allow the occlusion device to be advanced through a suitable delivery catheter)

In some embodiments, the anchor portion can comprise a plurality of anchor segments, wherein each of the plurality of anchor segments extend distally beyond the occluder portion when the occluder portion is in the occluder-deployed state and the anchor portion is in the anchor-deployed state. Each of the anchor segments can comprise a structure configured to enhance purchase of the anchor portion within the IPN or sIPN, such as a loop portion, a helical portion, a fin portion, a barb portion, or any combination thereof.

In some embodiments, the occluder portion can comprise a tissue growth member extending between the proximal end and the distal end of the occluder portion. In some cases, the tissue growth member can comprise one or more layers formed from an expanded polytetrafluoroethylene (ePTFE). These layers can form a proximal surface of the tissue growth member (i.e., facing the left atrium when the occlusion device is positioned within the ostium of the LAA).

Examples of suitable occlusion devices include those described in International Application Publication Nos. WO 2020/254907, WO 2013/067188, WO 2010/081041, WO 2010/148246, and WO 2009/052432, each of which is hereby incorporated by reference in its entirety.

By way of example, referring now to FIGS. 2A-2B, example occlusion device 20 and a distal end portion of a delivery system 22 are shown. The occlusion device 20 may include frame components of an occluder portion 24 and an anchor portion 26, the occluder portion 24 also including a tissue growth member 28 attached thereto. Further, the anchor portion 26 may be hingeably coupled to the occluder portion 24 such that the anchor portion 26 may be actuated, upon deployment of the occluder portion 24, between a deployed position and a non-deployed position (not shown) via an actuation mechanism at a handle (not shown) of the delivery system 22. With this arrangement, the occlusion device 20 and delivery system 22 may provide functionality of separating the steps of deploying the occluder portion 24 and the anchor portion 26, thereby, providing additional and enhanced functionality to the physician to properly position and implant the occlusion device 20 in the LAA.

As set forth, the occluder portion 24 may include an occluder material or a tissue growth member 28 attached thereto. The tissue growth member 28 may be a porous material, or other cell attaching material or substrate, configured to promote endothelization and tissue growth thereover. The tissue growth member 28 may extend over a proximal side of the medical device 20 and, particularly, over the occluder portion 24 and may extend over a portion of the anchor portion 26 and hinges coupling the anchor portion 26 to the occluder portion 24. As such, due to the shape of the frame components of the occluder portion 24, the tissue growth member 28 may include a proximal face that is generally convex to form an outer surface 40. The tissue growth member 28 may also include an inner surface 42 on its distal side that is generally concave shaped. In one embodiment, the tissue growth member 28 may extend primarily over an outside surface of frame components of the occluder portion 24 with a portion of the tissue growth member 28 extending on both the outside surface and the inside surface of the frame components of the occluder portion 24. In another embodiment, the tissue growth member 28 may extend primarily over both the outside surface and the inside surface of the frame components of the occluder portion 24 of the medical device 20. In another embodiment, the tissue growth member 28 may extend solely over the outside surface of the frame components of the occluder portion 24.

With respect to FIGS. 2B and 2C, the tissue growth member 28 may include one or more types of materials and/or layers. In one embodiment, the tissue growth member 28 may include a first material layer 30 and a second material layer 32. The first material layer 30 may primarily be an underside layer or base layer of the tissue growth member 28. The first material layer 30 may include porous and conformable structural characteristics. For example, the first material layer 30 may include a foam type material, such as, a polyurethane foam or any other suitable polymeric material, such as a polymer fabric, woven or knitted. The second material layer 32 may include one or more layers of, for example, an expanded polytetrafluoroethylene (ePTFE) material. The second material layer 32 may be attached to an outer surface of the first material layer 30 with, for example, an adhesive. In one embodiment, the second material layer 32 may include a first layer 32A, a second layer 32B, and a third layer 32C such that the first layer 32A may be directly attached to the first material layer 30 and the third layer 32C may be an outer-most layer covering the proximal side of the medial device 20 with the second layer 32B extending therebetween. The various layers of the second material layer 32 may be bonded together by adhesives and/or by a thermal bonding heat process or other appropriate processes known in the art. In one particular example, the outer-most layers, such as the second and third layers 32B, 32C, may be formed of an ePTFE material having an intemodal distance (sometimes referred to as pore size) of approximately 70μιη to approximately 90μιη. The first layer 32A of the second material layer 32, adjacent the first material layer 30, may be formed of an ePTFE material having a reduced internodal distance relative to the second and third layers 32B, 32C. For example, the internodal distance of the first layer 32A may be approximately IOptq. This first layer 32A may be bonded or adhered to the first material layer 30 using an adhesive material. Any other suitable sized layers of ePTFE may be employed, such as ePTFE having an internodal distance up to about 250μιη. Further, there may be one or more additional layers, similarly sized to the first layer 32A, extending over a hub end 34 with flaps 36 (outlined with an “X” configuration) where the delivery system 22 interconnects with the medical device 20 (see FIG. 2A).

The second material layer 32 made of ePTFE effectively prevents the passage of blood, due to the small internodal distance and pore size of the first layer 32A, while the larger internodal distance of other layers (e.g., 32B and 32C) enable tissue in-growth and endothelization to occur. Additionally, the first material layer 30, being formed of a polyurethane foam, enables aggressive growth of tissue from the LAA wall into the tissue growth member 28 at the inside or concave side of the medical device 20. Further, the first material layer 30 provides an exposed shelf 38 on the outer surface 40 around the periphery and distal end portion of the tissue growth member 28, which promotes aggressive fibroblast and tissue growth to further initiate endothelization over the outer surface 40 of the second material layer 32. It is noted that the use of appropriate adhesive materials between the first material layer 30 and the next adjacent layer 32A may also serve to fill in the pores of the next adjacent layer 32A and further inhibit possible flow of blood through the tissue growth member 28. Additional layers of ePTFE may also be included to the second material layer 32 of the tissue growth member 28.

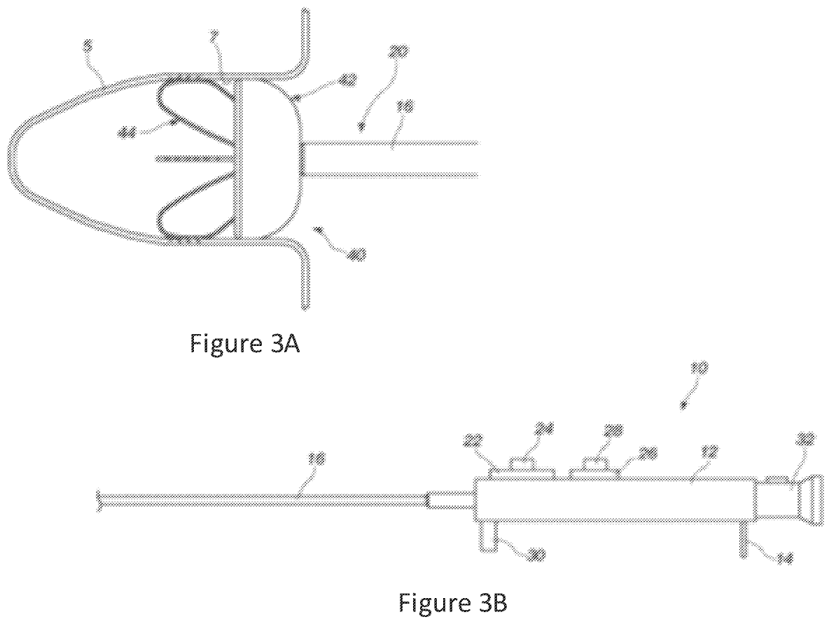

FIGS. 3A-3B illustrate another example occlusion device (40) that may be used in conjunction with the methods described herein. The occlusion device may be delivered by way of a delivery system 10 that includes a handle 12 with one or more actuators and a fluid port 14.

In addition, the system 10 may include a catheter 16 with a catheter lumen extending longitudinally therethrough and attached to a distal end of the handle 12. Such a catheter lumen may coincide and communicate with a handle lumen as well as communicate with the fluid port 14.

The actuators associated with the handle may be configured to actuate or move an occlusion device 40 disposed within a distal portion 20 of the catheter 16 to deploy the occlusion device 40 from or within the distal portion 20 of the catheter 16, to capture (or recapture) the occlusion device 40 within the distal portion 20 of the catheter, or to do both. Such an occlusion device 40 can be interconnected to the handle 12 via tethers coils or other structures or elements (generally referred to as tethers herein for convenience) extending through the catheter 16 (tethers not shown). For example, the tethers can have a proximal end connected to the handle 12 and a distal end thereof connected to the occlusion device 40. The occlusion device 40 can be manipulated to be deployed and recaptured at different stages by controlling movement of the tether/coils (via the actuators) and controlling movement of the catheter 16.

The occlusion device 40, shown in deployed position in FIG. 3A (wherein the device is fully or at least substantially expanded), may include an occluder portion 42 and an anchor portion 44. As briefly noted above, the occlusion device 40 can be controlled to deploy in discrete stages with one stage being the deployment of the occluder portion 42 and another, discrete stage being deployment of the anchor portion 44. In this manner, a physician can first deploy the occluder portion 42, locate a preferable position and orientation for the occluder portion 42 in the LAA 5 and, once positioned and oriented satisfactorily, the physician can maintain such position while independently deploying the anchor portion 44. As such, the occluder portion 42 and the anchor portion 44 are configured to be deployed independent of one another as discrete, affirmative acts by a physician or operator of the system 10.

As previously noted, the handle 12 may include multiple actuators including a release mechanism 32. The release mechanism 32 is configured to release the occlusion device 40 from the tethers once the occlusion device 40 is anchored in the LAA 5 as will be described in further detail below. Other actuators may include a first actuator 22, a second actuator 24, a third actuator 26, a fourth actuator 28 and a fifth actuator 30 as shown in FIG. 3A. For example, the first actuator 22 and the second actuator 24 may be configured to control movement of the occluder portion 42 while the third actuator 26 and the fourth actuator 28 may be configured to control movement of the anchor portion 44. The fifth actuator 30 may be configured to control maneuverability of the distal portion 20 of the catheter 16 to negotiate tight corners and facilitate orientation when placing the medical device 40 in the LAA 5. It should be noted that, for example, the first actuator 22 and the second actuator 24 can be configured as, or to act as, a single actuator for the occluder portion 42. Likewise, the third actuator 26 and the fourth actuator 28 can be configured as, or to act as, a single actuator for the anchor portion 44.

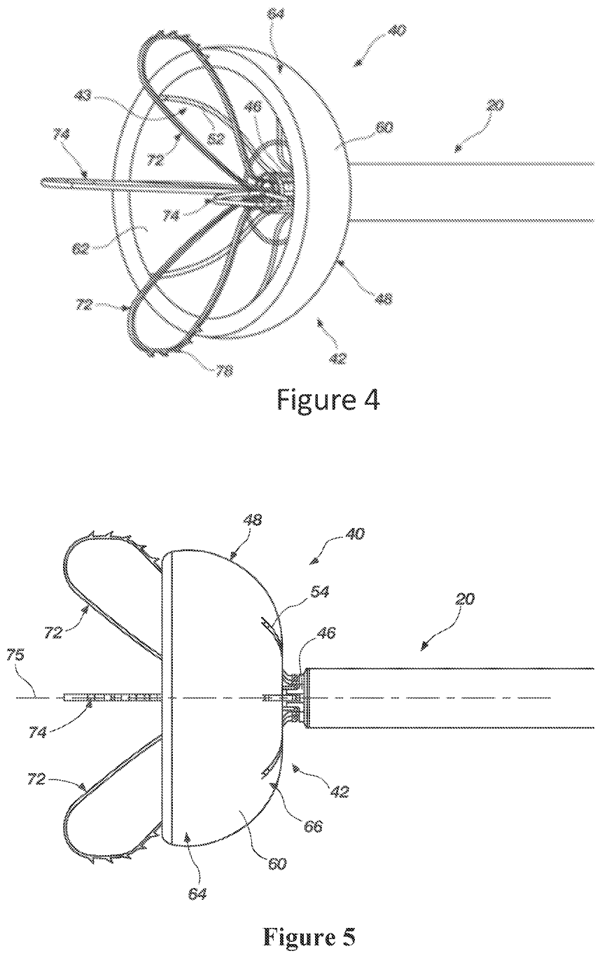

With reference to FIGS. 4 and 5, the occluder portion 42 may include an occluder frame 43 coupled to a hub 46 and a tissue growth member 48. The occluder frame 43 can include multiple occluder frame segments 50 extending radially and distally from the hub 46 generally in a spoke-like configuration. Such an occluder frame 43 is configured to assist in both expanding the tissue growth member 48 and in collapsing the tissue growth member 48. As such, each frame segment 50 may include an expander portion 52 and a collapser portion 54, wherein the expander portion 52 can include an overall length greater than that of the collapser portion 54. For example, each expander portion 52 may extend further radially, further distally, or both, as compared to a collapser portion 54.

Further, each frame segment 50 may include a clip 56 on each of the expander portion 52 and collapser portion 54. The clips 56 may be utilized to attach the tissue growth member 48 between the expander portion 52 and the collapser portion 54.

The tissue growth member 48 may include a porous structure configured to induce or promote tissue in-growth, or any other suitable structure configured to promote tissue in-growth. The tissue growth member 48 can include, for example, a body or a structure exhibiting a cup-like shape having an outer surface 60 and an inner surface 62. The outer surface 60 may include a distal surface portion 64 and a proximal surface portion 66. The outer surface distal surface portion 64 of the tissue growth member 48 can be sized and configured to be in direct contact with a tissue wall 7 within the LAA 5. In one embodiment, the tissue growth member 48 may be configured to self expand from a confined or constricted configuration to an expanded or deployed configuration. In one embodiment, the tissue growth member 48 may include a polymeric material, such as polyurethane foam. Other materials with desired porosity can also be used, such as felt, fabric, Dacron®, Nitinol braded wire, or polymeric or Nitinol felt. In the case of foam, such foam may be a reticulated foam, typically undergoing a chemical or heating process to open the pores within the foam as known by those of ordinary skill in the art. The foam may also be a non-reticulated foam. In one embodiment, the foam may include graded density or a graded porosity, as desired, and manipulated to expand in a desired shape when the frame member is moved to the expanded configuration.

In another embodiment, the tissue growth member 48 may include polyurethane foam with a skin structure on the inner surface 62, on the outer surface 60, or on both surfaces. For example, a skin structure may be formed on the inner surface 62 and be configured to inhibit blood from flowing through the tissue growth member 48, while the outer surface 60 of the tissue growth member may be configured to receive blood cells within its pores and induce tissue in-growth. In one embodiment, such a skin structure can include a layer of material, such as tantalum, sputtered to a surface of the tissue growth member 48. In another embodiment, the skin structure can include a polyurethane foam skin. Another example includes attaching expanded polytetrafluoroethylene (ePTFE) to the outer surface 60 or inner surface 62 of the tissue growth member 48, the ePTFE having minimal porosity to substantially inhibit blood flow while still allowing endothelization thereto.

In one embodiment, the anchor portion 44 may include a plurality of anchor segments and an anchor hub system 70. The anchor hub system 70 may be configured to be positioned and disposed within or adjacent to the hub 46. The plurality of anchor segments can include, for example, a first anchor segment 72 and a second anchor segment 74. Each of the first anchor segment 72 and the second anchor segment 74 may include a pedal or loop configuration (shown here in an expanded configuration), with, for example, two loop configurations for each of the first and second anchor segments 72 and 74, that are interconnected together via the anchor hub system 70. Each loop may be substantially oriented orthogonally with respect to an adjacent loop (i.e., in the embodiment shown in FIGS. 4 and 5, each loop of anchor component 72 being orthogonal to adjacent loops of anchor component 74). It is noted that, as used herein, the term “loop” does not require that a closed curve be formed of the component, but rather that a substantially closed curved or an open curve having a portion of the curve return on itself may also be considered as a “loop.”

While in the expanded configuration, each loop may extend distally of the occluder portion 42 and radially outward to a larger configuration than the anchor hub system 70. In other words, at least a portion of the anchor segments 72 and 74 extend distally beyond the distal-most portion of the occluder portion 42 and radially beyond the radial-most portion of the occluder portion as taken from a longitudinal axis 75 extending through the hub system 70. Each loop of an anchor segment 72 and 74 may also include engagement members or traction nubs 78 on an outer periphery of a loop configuration, the traction nubs 78 being sized and configured to engage and grab a tissue wall 7 and/or the IPN or sIPN within the LAA 5.

Each of the loop configurations of the first anchor segment 72, while in an expanded configuration, are substantially co-planar with each other and in a substantially flat configuration. Likewise, each of the loop configurations of the second anchor segment 74, while in an expanded configuration, are substantially co-planar with each other and in a substantially flat configuration. In one embodiment, the first anchor segment 72 may be attached to the second anchor segment 74 such that the loop configuration between the first and second anchor segments 72 and 74 are oriented substantially orthogonal with respect to each other. In other words, the plane in which the first anchor segment 72 is positioned or oriented is substantially orthogonal with respect to the plane of the second anchor segment 74. In other embodiments, there may be more than two anchor segments, in which case such anchor segments may or may not be oriented in a substantially orthogonal manner relative to each other.

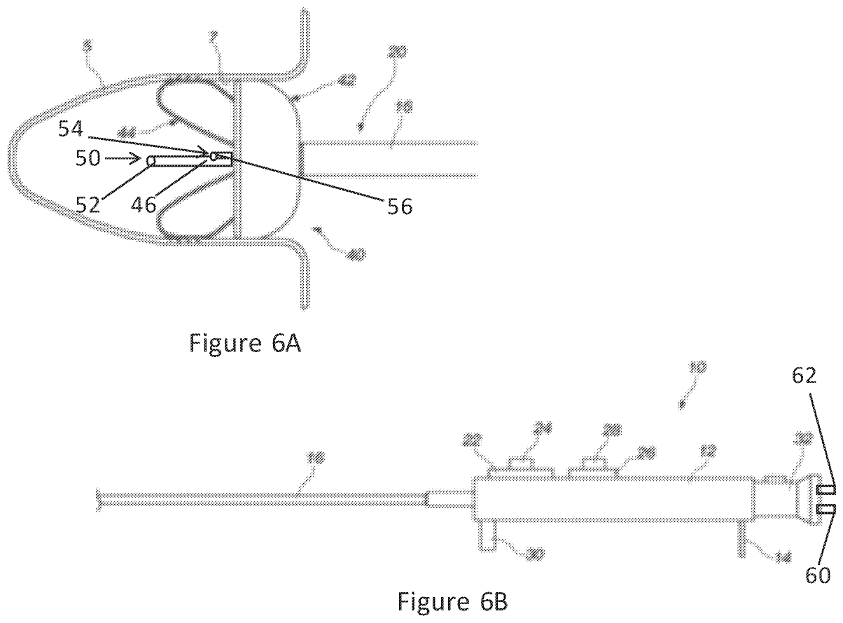

FIGS. 6A-6B illustrate another example occlusion device (40) that may be used in conjunction with the methods described herein. The occlusion device may be delivered by way of a delivery system 10 that includes a handle 12 with one or more actuators and a fluid port 14. In addition, the system 10 may include a catheter 16 with a catheter lumen extending longitudinally therethrough and attached to a distal end of the handle 12. Such a catheter lumen may coincide and communicate with a handle lumen as well as communicate with the fluid port 14.

The actuators associated with the handle may be configured to actuate or move an occlusion device 40 disposed within a distal portion 20 of the catheter 16 to deploy the occlusion device 40 from or within the distal portion 20 of the catheter 16, to capture (or recapture) the occlusion device 40 within the distal portion 20 of the catheter, or to do both. Such an occlusion device 40 can be interconnected to the handle 12 via tethers coils or other structures or elements (generally referred to as tethers herein for convenience) extending through the catheter 16 (tethers not shown). For example, the tethers can have a proximal end connected to the handle 12 and a distal end thereof connected to the occlusion device 40. The occlusion device 40 can be manipulated to be deployed and recaptured at different stages by controlling movement of the tether/coils (via the actuators) and controlling movement of the catheter 16.

The occlusion device 40, shown in deployed position in FIG. 3A (wherein the device is fully or at least substantially expanded), may include an occluder portion 42 and an anchor portion 44. As briefly noted above, the occlusion device 40 can be controlled to deploy in discrete stages with one stage being the deployment of the occluder portion 42 and another, discrete stage being deployment of the anchor portion 44. In this manner, a physician can first deploy the occluder portion 42, locate a preferable position and orientation for the occluder portion 42 in the LAA 5 and, once positioned and oriented satisfactorily, the physician can maintain such position while independently deploying the anchor portion 44. As such, the occluder portion 42 and the anchor portion 44 are configured to be deployed independent of one another as discrete, affirmative acts by a physician or operator of the system 10.

The occluder portion 42 comprises a proximal end and a distal end, the proximal end being coupled to a hub 46 having an injection lumen 50 passing axially therethrough. Injection lumen 50 terminates distally at an injection outlet 52. The delivery catheter can further include an injection channel 60 extending from the proximal end to the distal end and terminating in an outlet opening (not shown) which is fluidly connected to injection lumen 50 of the occlusion device. The injection channel and injection lumen provide a fluid flow path, allowing for the physician to inject a photocurable biomaterial into the LAA after the occlusion device has been deployed within the ostium of the LAA.

Optionally, hub 46 can further comprise a second lumen 54 passing axially therethrough which is fluidly isolated from injection lumen 50. The second lumen 54 can terminate distally at a fluid inlet 56. In some cases, the injection outlet 52 can be separated from and distal to the fluid inlet 56. In these embodiments, the delivery catheter can further comprise an auxiliary lumen 62 fluidly isolated from the at least one injection channel and extending from the proximal end to the distal end and terminating in an auxiliary opening (not shown) which is fluidly connected to the second lumen of the occlusion device. The auxiliary lumen and second lumen provide a second fluid flow path, allowing for the physician to, for example, withdraw blood from the LAA after the occlusion device has been deployed within the ostium of the LAA. The auxiliary lumen and second lumen provide a second fluid flow path, allowing blood present in the LAA to flow from the LAA into the second lumen when a photocurable biomaterial is injected into the LAA through the injection channel of the delivery catheter body and the injection lumen.

As previously noted, the handle 12 may include multiple actuators including a release mechanism 32. The release mechanism 32 is configured to release the occlusion device 40 from the tethers once the occlusion device 40 is anchored in the LAA 5 as will be described in further detail below. Other actuators may include a first actuator 22, a second actuator 24, a third actuator 26, a fourth actuator 28 and a fifth actuator 30 as shown in FIG. 3A. For example, the first actuator 22 and the second actuator 24 may be configured to control movement of the occluder portion 42 while the third actuator 26 and the fourth actuator 28 may be configured to control movement of the anchor portion 44. The fifth actuator 30 may be configured to control maneuverability of the distal portion 20 of the catheter 16 to negotiate tight corners and facilitate orientation when placing the medical device 40 in the LAA 5. It should be noted that, for example, the first actuator 22 and the second actuator 24 can be configured as, or to act as, a single actuator for the occluder portion 42. Likewise, the third actuator 26 and the fourth actuator 28 can be configured as, or to act as, a single actuator for the anchor portion 44.

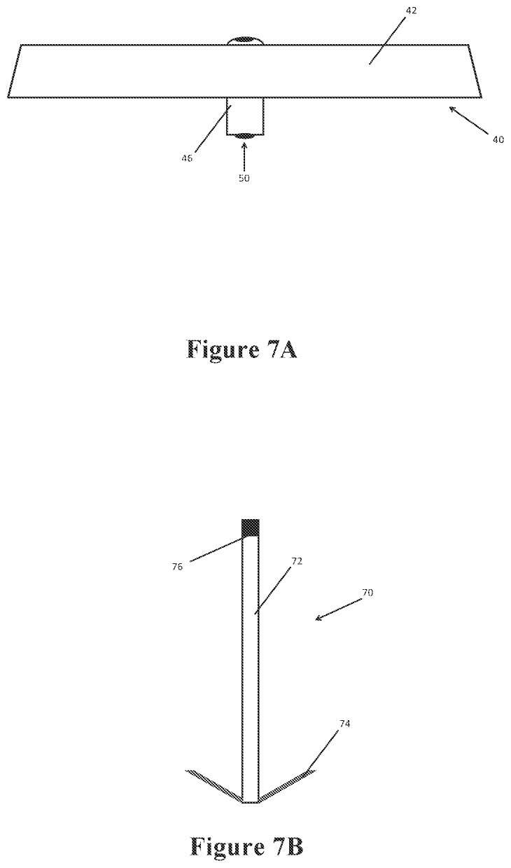

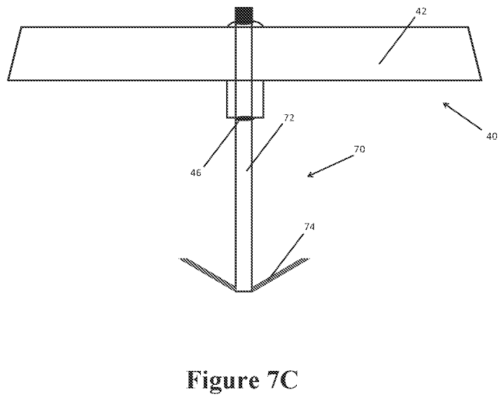

FIGS. 7A-7C illustrate another example occlusion device 40. As shown in FIG. 7A, the occlusion device can include an occluder portion 42 comprising a proximal end and a distal end, the proximal end coupled to a hub 46 having an injection lumen 50 passing axially therethrough. In these embodiments, the occlusion device does not include an integrated anchor portion, though one may optionally be present. As shown in FIG. 7B, the occlusion device can include a separate attachable anchor portion 70. The anchor portion can include one or more anchor segments 72 coupled to a fastener (76) that allows the anchor portion to be coupled (e.g., reversibly) to the occlusion portion. As shown in FIG. 7C, in some embodiments, the anchor portion can be coupled to the occluder portion 42 by way of the hub 46 by means of fastener 76, such as by screwing the anchor portion to the hub. As with other embodiments described above, the anchor portion can be configured to move between an anchor-deployed state and an anchor-nondeployed state. For example, the anchor portion can include one or more barbs, fins, etc. (74) which can be deployed once the anchor portion has been advanced through the injection lumen and coupled to the occluder portion. The anchor portion (including the fastener) can be structured such that coupling of the anchor portion to the occlude portion seals any lumens which pass axially through the hub.

In other embodiments, the occlusion device can comprise an existing approved implant for occluding the LAA (e.g., the WATCHMAN™ LAAC Implant, the PLAATO (percutaneous left atrial appendage transcatheter occlusion) implant, or the Amplatzer device), optionally modified to include an anchor portion to allow them to more strongly interact and be secured by a biocompatible polymeric matrix formed in the LAA.

In some methods, the delivery catheter(s) can comprise an element configured to irradiate the photocurable biomaterial with actinic radiation, such as a water light pipe, light source (e.g., LED), or a combination thereof.

These example occlusion devices are discussed to exemplify some of the characteristics of suitable occlusion devices. One of ordinary skill in the art will appreciate that other occlusion devices having the characteristics above can also be used.

### Methods of Occluding the LAA

The photocurable biomaterial and the occlusion device can be delivered to the LAA percutaneously (e.g., using a delivery catheter assembly and delivery system described below). The particular components and features of the delivery catheter assembly and delivery system can vary based on a number of factors, including the nature of the photocurable biomaterial to be delivered. For example, the number of lumens in the delivery catheter assembly and/or the presence or absence of a mixing channel can be selected in view of the identity of the photocurable biomaterial.

Examples of suitable delivery systems include those described in International Application Publication Nos. WO 2020/254907, WO 2013/067188, WO 2010/081041, WO 2010/148246, and WO 2009/052432, each of which is hereby incorporated by reference in its entirety. Suitable delivery systems are also described above and exemplified, for example, in FIGS. 3A-3B and FIGS. 6A-6B.

In some embodiments, the delivery system can comprise a delivery catheter that includes a catheter body extending between a proximal end and a distal end, the catheter body comprising a wall structure that defines at least one injection channel extending from the proximal end to the distal end and terminating in an outlet opening; a handle coupled to the proximal end of the delivery catheter body, and the occlusion device operatively coupled to the handle and coupled to the distal end of the delivery catheter body. In embodiments where the occlusion device includes an injection lumen and the delivery catheter includes an injection channel, the injection channel can be fluidly connected to the injection lumen of the occlusion device. In embodiments where the occlusion device includes a second lumen and the delivery catheter includes an auxiliary lumen, the auxiliary lumen can be fluidly connected to the second lumen of the occlusion device.

Optionally, the delivery system can further comprise a sheath having a proximal end portion, a distal end portion having a distal tip, and a wall circumferentially enclosing a sheath lumen extending along an entire length of the sheath. The delivery catheter can be sized to be received and selectively advanceable within the sheath lumen such that the occlusion device can be passed through the sheath lumen to a position distal of the distal tip. Optionally, the sheath can further comprise at least one inflation channel within the wall of the sheath; and a balloon coupled to the distal end portion of the sheath and positioned in fluid communication with the at least one inflation channel of the sheath, the balloon enclosing an interior space. The wall of the delivery catheter body can define at least one outlet opening to provide fluid communication between the at least one inflation channel and the interior space of the balloon (allowing for inflation of the balloon using a suitable fluid).

Another example delivery catheter assembly can be based on the delivery catheter assembly described in International Application Publication No. WO 2019/099686, which is hereby incorporated by reference in its entirety. Briefly, the catheter assembly can comprise a first catheter body having a proximal end portion, a distal end portion having a distal tip, and a wall that circumferentially encloses a primary opening. The first catheter body can further comprise at least one inflation channel within the wall of the first catheter body. The primary opening of the first catheter body can extend along an entire length of the first catheter body. The catheter assembly can also comprise a first balloon coupled to the distal end portion of the first catheter body and positioned in fluid communication with the at least one inflation channel of the first catheter body. The first balloon can enclose an interior space, and the first catheter body can extend through the interior space of the first balloon in a proximal-to-distal direction such that at least the distal tip of the first catheter body is positioned distal of the first balloon.

The catheter assembly can also comprise a second catheter body partially received within the primary opening of, and selectively moveable relative to, the first catheter body. The second catheter body can include a proximal end portion, a distal end portion having a tip, and a wall that circumferentially encloses a primary opening. The second catheter body can further comprise at least one inflation channel within the wall of the second catheter body. The primary opening of the second catheter body can extend along an entire length of the second catheter body. The catheter assembly can further include a second balloon coupled to the distal end portion of the second catheter body and positioned in fluid communication with the at least one inflation channel of the second catheter body. The second balloon can enclose an interior space, and the second catheter body can extend through the interior space of the second balloon in the proximal-to-distal direction such that at least the distal tip of the second catheter body is positioned distal of the second balloon.

Additionally, the catheter assembly can comprise a third catheter body partially received within the primary opening of, and selectively moveable relative to, the second catheter body. The third catheter body can include a proximal end portion, a distal end portion, and a wall structure that defines at least one injection channel extending from the proximal end portion toward the distal end portion. The distal end portion of the third catheter body can further comprise at least one outlet opening positioned in fluid communication with the at least one injection channel. The third catheter body can be removable from the primary opening of the second catheter.

In these embodiments, the occlusion device can be positioned within the ostium of the LAA using a delivery system, wherein the delivery system comprises: a delivery catheter body extending between a proximal end and a distal end, a handle coupled to the proximal end of the delivery catheter body, and the occlusion device operatively coupled to the handle and coupled to the distal end of the delivery catheter body. The delivery system can be sized to be received within the primary opening of, and selectively moveable relative to, the second catheter body such that the occlusion device can be passed through the primary opening of the second catheter body to a position distal of the second balloon. The delivery system can be similar to those described in International Application Publication Nos. WO 2020/254907, WO 2013/067188, WO 2010/081041, WO 2010/148246, and WO 2009/052432, each of which is hereby incorporated by reference in its entirety. Suitable delivery systems are also described above and exemplified, for example, in FIGS. 3A-3B and FIGS. 6A-6B.

Such delivery catheters can be utilized in procedures where the occlusion device is positioned within the ostium of the LAA after injection of the photocurable biomaterial into the LAA.

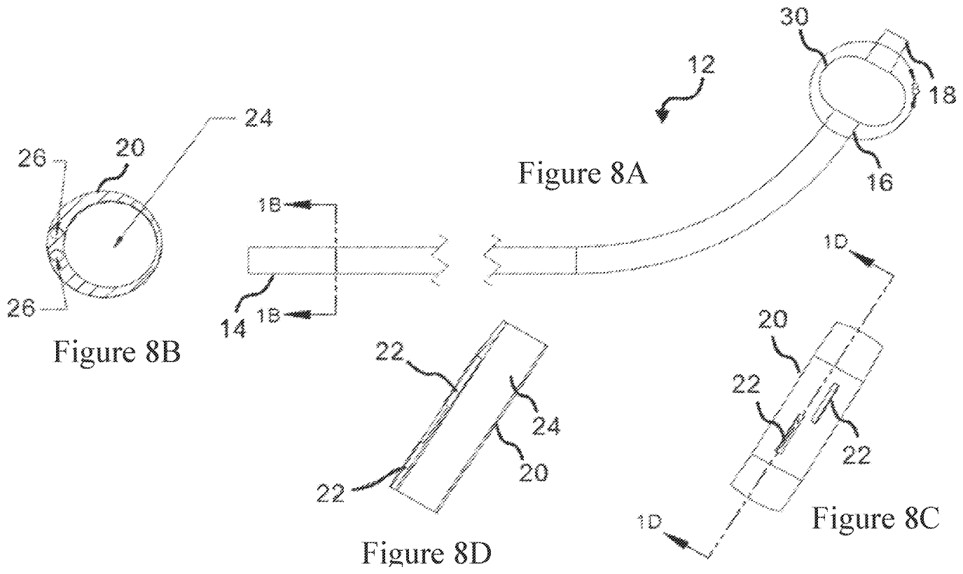

Referring now to FIGS. 8A-10D, the catheter assembly 10 can comprise three catheter bodies 12, 40, 70 and two balloons 30, 60 which cooperate with each other to deliver at least one photocurable biomaterial and an occlusion device to the LAA of a subject's heart in a manner such that complete closure of the LAA is achieved.

In exemplary aspects, as shown in FIG. 8A, the catheter assembly 10 can comprise a first catheter body 12 that includes a proximal end portion 14, a distal end portion 16 having a distal tip 18, and a wall 20 that circumferentially encloses a primary opening 22. In these aspects, the primary opening 22 of the first catheter body 12 can extend along an entire length of the first catheter body. As shown in FIG. 8B, the first catheter body 12 can comprise at least one inflation channel 26 (optionally, a plurality of inflation channels, such as, for example, two inflation channels) within the wall 20 of the first catheter body. In further aspects, the catheter assembly 10 can comprise a first balloon 30 that can be coupled to the distal end portion 16 of the first catheter body 12 and positioned in fluid communication with the at least one inflation channel 26 of the first catheter body. In these aspects, the first balloon 30 can enclose an interior space 32. As shown in FIGS. 8C and 8D, the wall 20 of the first catheter body 12 can define at least one outlet opening 22 (optionally, a plurality of outlet openings) to provide fluid communication between the at least one inflation channel 26 and the interior space 32 of the first balloon 30. In some aspects, the each outlet opening can be in fluid communication with a respective inflation channel. In additional aspects, the plurality of outlet openings can be circumferentially spaced, axially spaced (in a distal or proximal direction), or both circumferentially and axially spaced in a staggered configuration In further aspects, the first catheter body 12 can extend through the interior space 32 of the first balloon 30 in a proximal-to-distal direction such that at least the distal tip 18 of the first catheter body 12 is positioned distal of the first balloon 30.

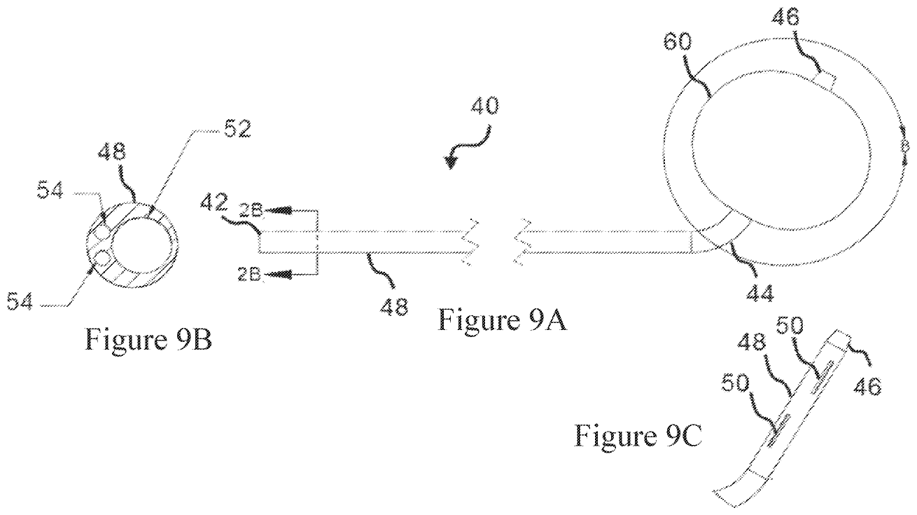

Referring to FIGS. 9A-9C, the catheter assembly 10 can comprise a second catheter body 40 that can be partially received within the primary opening 24 of, and selectively moveable relative to, the first catheter body 12. Optionally, in some aspects, it is contemplated that the second catheter body 40 can be selectively retractable relative to the first catheter body 12. In further optional aspects, it is contemplated that the first and second catheter bodies 12, 40 can be selectively lockable to maintain a desired position and orientation of the second catheter body 40 relative to the first catheter body 12. Optionally, in exemplary aspects, it is contemplated that the proximal ends of the first and second catheter bodies 12, 40 can be provided with Tuohy-type locking mechanisms as are known in the art (e.g., Tuohy-Borst adapters) to use friction to lock the first catheter body to the second catheter body. However, it is contemplated that any suitable locking mechanism as is known in the art can be used for this purpose. In additional aspects and as shown in FIG. 9A, the second catheter body 40 can include a proximal end portion 42, a distal end portion 44 having a distal tip 46, and a wall 48 that circumferentially encloses a primary opening 52 of the second catheter body 40. The primary opening 52 of the second catheter body 40 can extend along an entire length of the second catheter body. As shown in FIG. 9B, the second catheter body 40 can further comprises at least one inflation channel 54 (optionally, a plurality of inflation channels, such as for example, two inflation channels) within the wall 48 of the second catheter body 40.

In further aspects, the catheter assembly 10 can comprise a second balloon 60 that can be coupled to the distal end portion 44 of the second catheter body 40 and positioned in fluid communication with the at least one inflation channel 54 of the second catheter body. In these aspects, the second balloon 60 can enclose an interior space 62. As shown in FIG. 9C, the wall 48 of the second catheter body 40 can define at least one outlet opening 50 (optionally, a plurality of outlet openings) to provide fluid communication between the at least one inflation channel 54 and the interior space 62 of the second balloon. In some aspects, the each outlet opening 50 can be in fluid communication with a respective inflation channel 54. In additional aspects, the plurality of outlet openings 50 can be circumferentially spaced, axially spaced (in a distal or proximal direction), or both circumferentially and axially spaced in a staggered configuration. In further aspects, the second catheter body 40 can extend through the interior space 62 of the second balloon 60 in the proximal-to-distal direction such that at least the distal tip 46 of the second catheter body 40 is positioned distal of the second balloon 60.

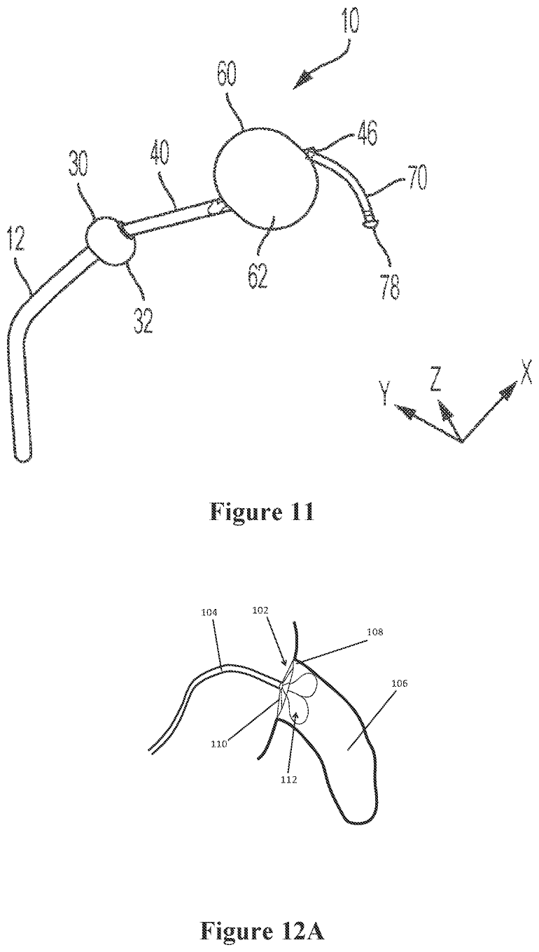

As depicted in FIG. 11, it is contemplated that when in an inflated position (e.g., a fully inflated position), the second balloon 60 can be larger (e.g., have a larger diameter) than the first balloon 30 (when the first balloon is also in an inflated or fully inflated position). Optionally, in exemplary aspects, it is contemplated that the maximum inflated diameter of the first balloon 30 can range from about 10 mm to about 20 mm or, more preferably, be about 15 mm. Optionally, in additional aspects, it is contemplated that the maximum inflated diameter of the second balloon 60 can range from about 30 mm to about 50 mm or from about 35 mm to about 45 mm or, more preferably, be about 40 mm. Optionally, in further exemplary aspects, it is contemplated that the first and second balloons are not spherical. For example, it is contemplated that each balloon can have an axial length (relative to the length of the catheter bodies) that is less than its maximum inflated diameter.

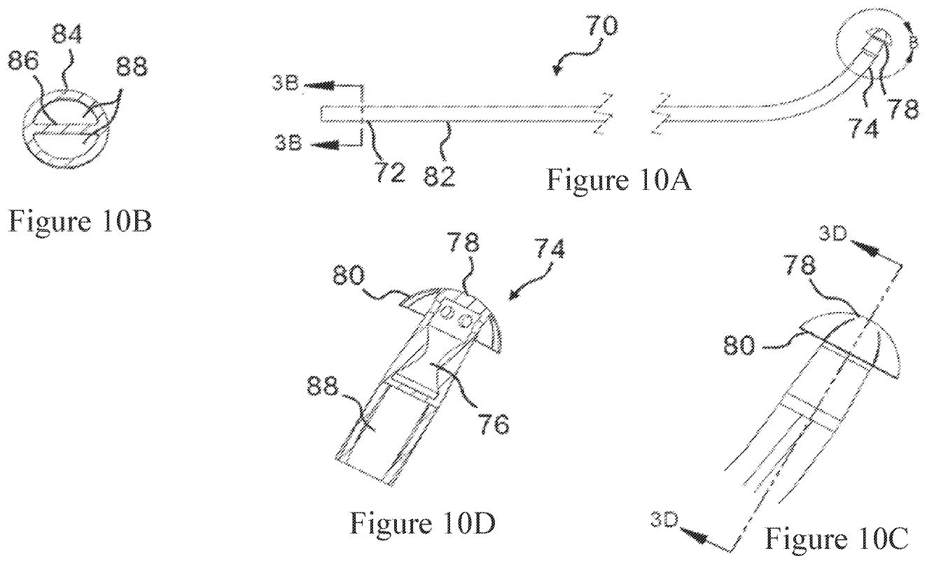

Referring to FIGS. 10A-10D, the catheter assembly 10 can comprise a third catheter body 70 that can be partially received within the primary opening 52 of, and selectively moveable relative to, the second catheter body 40. Optionally, in some aspects, the third catheter body 70 can be selectively retractable relative to the second catheter body 40. It is contemplated that, when the third catheter body is fully retracted, the third catheter body 70 can be fully received within the primary opening 52 of the second catheter body 40. In these aspects, as shown in FIG. 10A, the third catheter body 70 can have a proximal end portion 72, a distal end portion 74, and a wall structure 82 that defines at least one injection channel 88 extending from the proximal end portion 72 toward the distal end portion 74. Optionally, in some aspects, the at least one injection channel 88 can comprise a single injection channel. Alternatively, in some aspects, the at least one injection channel 88 of the third catheter body 70 can comprise a plurality of injection channels. For example, in some aspects, the at least one injection channel 88 of the third catheter body 70 can comprise first and second injection channels 88. In these aspects, as shown in FIG. 10B, the wall structure 82 of the third catheter body 70 can comprise an outer wall 84 and an inner wall 86 that extends between opposing portions of the outer wall to define the first and second injection channels 88. As shown in FIG. 10D, the distal end portion 74 of the third catheter body 70 can further comprise at least one outlet opening 90 positioned in fluid communication with the at least one injection channel 88. Optionally, in some aspects, the at least one outlet opening 90 of the distal end portion 74 of the third catheter body 70 can comprise a plurality of outlet openings. In further optional aspects, the distal end portion 74 of the third catheter body 70 can comprises a static mixing component 76 positioned between the at least one injection channel 88 and the at least one outlet opening 90, as shown in FIG. 10D. As used herein, the term “static mixing component” does not require any particular structural arrangement. Rather, the “static mixing component” includes any in-line structure that promotes mixing of the materials delivered through the respective injection channels 88 as further disclosed herein. Optionally, the static mixing component 76 can be a housed-elements type static mixer, a plate-type static mixer, or combinations thereof. More generally, it is contemplated that the static mixing component 76 can have a central receiving channel that provides for a variable flow pathway between the at least one injection channel 88 and the at least one outlet opening 90. Such a variable flow pathway can be created by projections and recesses (changes in diameter) of the interior surfaces of the static mixing component, as well as the presence of obstructions that prevent portions of the injected materials from following a consistent axial path in a proximal-to-distal direction. It is understood that when only a single injection channel 88 is provided, or in other situations where mixing of injectable components is unnecessary prior to delivery, it is possible to omit the static mixing component from the third catheter body 70.

In further aspects, the distal end portion 74 of the third catheter body 70 can have a distal tip 78 and a diaphragm 80 that is secured to the distal tip. In these aspects, the diaphragm 80 can extend outwardly from the distal tip 78. It is contemplated that the diaphragm 80 of the third catheter body 70 can occlude the primary opening 52 of the second catheter body 40 to prevent entry of material into the primary opening of the second catheter body in a distal-to-proximal direction. For example, it is contemplated that the diaphragm can be biased and/or deformable to a blocking position in which the outer diameter of the diaphragm is greater than the diameter of the primary opening 52 of the second catheter body. It is further contemplated that the diaphragm 80 can comprise a flexible material that is deformable as the third catheter body 70 exits the second catheter body (upon initial deployment) or is received within the second catheter body (upon retraction of the third catheter body), with the diaphragm blocking the entry of material into the second catheter body. In exemplary aspects, and as shown in FIGS. 10A-10C, the diaphragm can be secured to the distal tip 78 and have a convex outer surface extending circumferentially around the distal tip, with a proximal portion of the diaphragm at least partially overlapping with the outlet openings 90 (moving in a distal-to-proximal direction). As the third catheter body continues to move in a proximal direction within the primary opening of the second catheter body, the proximal surface of the diaphragm can contact the portions of the second catheter body to thereby movement of the diaphragm to a fully blocking position. Prior to complete receipt of the third catheter body 70 within the primary opening 52 of the second catheter body 40, it is contemplated that the injection of material through the outlet openings of the third catheter body can displace other fluid within the delivery site (e.g., LAA), with the displaced fluid flowing into the primary opening of the second catheter body.

It is contemplated that the first, second, and third catheter bodies can be formed from a variety of materials. The materials can be selected such that the first, second, and third catheter bodies have structural integrity sufficient to permit advancement of each catheter body as described herein and permit maneuvering and operation of each catheter body, while also permitting yielding and bending in response to encountered anatomical barriers and obstacles within the subject's body (e.g., within the vasculature). In exemplary aspects, the first, second, and/or third catheter bodies can be formed front a material or combination of materials, such as polymers, metals, and polymer-metal composites. In some aspects, soft durometer materials can be used to form all or part of the respective catheter body to reduce discomfort and minimize the risk of damage to the subject's vasculature (e.g., perforation). In some embodiments, the first, second, and/or third catheter bodies can be formed, in whole or in part, from a polymeric material. Examples of suitable plastics and polymeric materials include, but are not limited to, silastic materials and silicon-based polymers, polyether block amides (e.g., PEBAX®, commercially available from Arkema, Colombes, France), polymide, polyurethanes, polyamides (e.g., Nylon 6,6), polyvinylchlorides, polyesters e.g., HYTREL®, commercially available from DuPont, Wilmington, Del.), polyethylenes (PE), polyether ether ketone (PEEK), fluoropolymers such as polytetrafluoroethylene (PTFE), perfluoroalkoxy, fluorinated ethylene propylene, or blends and copolymers thereof. Examples of suitable metals which can form some or all of the first, second, and/or third catheter bodies include stainless steel (e.g., 304 stainless steel), nickel and nickel alloys (e.g., nitinol or MP-35N), titanium, titanium alloys, and cobalt alloys. In certain embodiments, each catheter body can comprise two different materials. Radiopaque alloys, such as platinum and titanium alloys, may also be used to fabricate, in whole or in part, the delivery catheter to facilitate real-time imaging during procedures performed using the delivery catheter. Optionally, the first, second, and/or third catheter bodies can be coated or treated with various polymers or other compounds in order to provide desired handling or performance characteristics, such as to increase lubricity. In certain embodiments, the first, second, and/or third catheter bodies can be coated with polytetrafluoroethylene (PTFE) or a hydrophilic polymer coating, such as poly(caprolactone), to enhance lubricity and impart desirable handling characteristics.

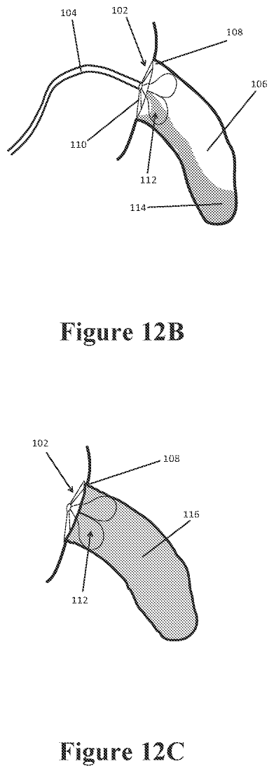

An example method for occluding the LAA of a patient is illustrated in FIGS. 12A-12C. These methods can comprise advancing a delivery system percutaneously through the patient's vasculature to reach the patient's right atrium. The delivery system can comprise a delivery catheter, wherein the delivery catheter comprises: a delivery catheter body extending between a proximal end and a distal end, the catheter body comprising a wall structure that defines at least one injection channel extending from the proximal end to the distal end and terminating in an outlet opening; a handle coupled to the proximal end of the delivery catheter body, and an occlusion device operatively coupled to the handle and coupled to the distal end of the delivery catheter body, wherein the occlusion device comprises an occluder portion comprising a proximal end and a distal end, the proximal end coupled to a hub having an injection lumen passing axially therethrough; and an anchor portion operably coupled to the occluder portion, wherein the outlet opening of the at least one injection channel is fluidly connected to the injection lumen of the occlusion device. Next, the delivery system can be advanced through an opening in the interatrial septum to reach the patient's left atrium. As shown in FIG. 12A, the occlusion device 102 can then be deployed within the ostium of the LAA 108 using the delivery catheter 104, such that the anchor portion 112 extends into the internal volume of the LAA (106). When deployed, the occluder portion 110 of the occlusion device 102 can substantially isolate the internal volume of the LAA (106) from the left atrium. Next, as shown in FIG. 12B, a photocurable biomaterial 114 can be injected into the LAA through the injection channel of the delivery catheter body and the injection lumen of the occlusion device 102. The photocurable biomaterial can be flowable and conform to the internal anatomy of the LAA prior to crosslinking. Subsequently, methods can involve irradiating the photocurable biomaterial with actinic radiation, thereby inducing crosslinking of the photocurable biomaterial in sittu in the LAA to form the IPN or sIPN that fills and occupies the internal volume of the LAA. The occlusion device can be retained within the ostium of the LAA until irradiation and photocrosslinking have occurred and the photocurable biomaterial has solidified to form the IPN or sIPN. At this point, delivery catheter 104 can be withdrawn, leaving the occlusion device in place. As shown in FIG. 12C, because of the compliant nature of the photocurable biomaterial, the resulting IPN or sIPN (116) can be interpenetrated by both the anchor portion (112) of the occlusion device and trabeculae present in the LAA (not shown for simplicity). In this way, the IPN or sIPN ensures that the occlusion device is retained within the ostium of the LAA 108. Further, the IPN or sIPN can function as an occlusive body, filling and occupying the internal volume of the LAA without adversely impacting cardiac function. In addition, the occlusion device can isolate the IPN or sIPN from blood present in the left atrium, provide a scaffold for endothelialization, of a combination thereof.

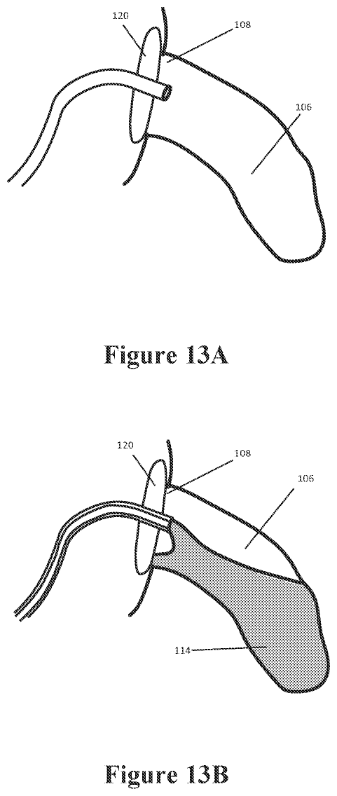

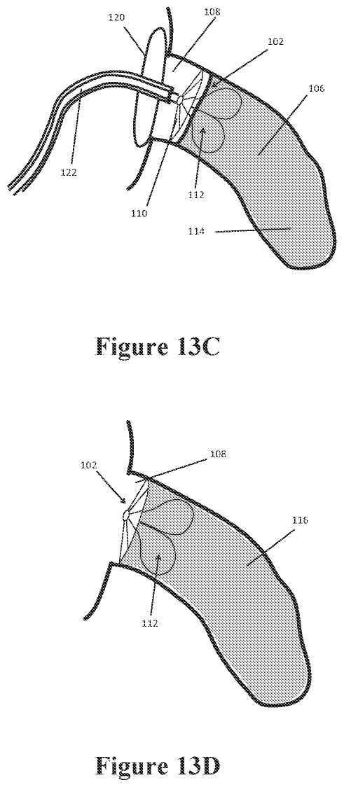

An alternative method for occluding the LAA of a patient is illustrated in FIGS. 13A-13D. These methods can comprise advancing a delivery catheter assembly percutaneously through the patient's vasculature to reach the patient's right atrium. The delivery catheter assembly can comprise: a first catheter body having a proximal end portion, a distal end portion having a distal tip, and a wall that circumferentially encloses a primary opening, wherein the first catheter body further comprises at least one inflation channel within the wall of the first catheter body, wherein the primary opening of the first catheter body extends along an entire length of the first catheter body; a first balloon coupled to the distal end portion of the first catheter body and positioned in fluid communication with the at least one inflation channel of the first catheter body, the first balloon enclosing an interior space, wherein the first catheter body extends through the interior space of the first balloon in a proximal-to-distal direction such that at least the distal tip of the first catheter body is positioned distal of the first balloon; a second catheter body partially received within the primary opening of, and selectively moveable relative to, the first catheter body, wherein the second catheter body has a proximal end portion, a distal end portion having a tip, and a wall that circumferentially encloses a primary opening, wherein the second catheter body further comprises at least one inflation channel within the wall of the second catheter body, wherein the primary opening of the second catheter body extends along an entire length of the second catheter body; a second balloon coupled to the distal end portion of the second catheter body and positioned in fluid communication with the at least one inflation channel of the second catheter body, the second balloon enclosing an interior space, wherein the second catheter body extends through the interior space of the second balloon in the proximal-to-distal direction such that at least the distal tip of the second catheter body is positioned distal of the second balloon; and a third catheter body partially received within the primary opening of, and selectively moveable relative to, the second catheter body, wherein the third catheter body has a proximal end portion, a distal end portion, and a wall structure that defines at least one injection channel extending from the proximal end portion toward the distal end portion, wherein the distal end portion of the third catheter body further comprises at least one outlet opening positioned in fluid communication with the at least one injection channel. Next, the delivery catheter assembly can be advanced through an opening in the interatrial septum to reach the patient's left atrium. The first balloon can then be inflated to anchor and secure the delivery catheter assembly within the left atrium. Next, the second catheter body can be advanced relative to the first catheter body to reach the LAA. As shown in FIG. 13A, the second balloon 120 can then be inflated to occlude the ostium of the LAA (108) of the patient. The third catheter body can then be advanced relative to the second catheter body. Next as shown in FIG. 13B, a photocurable biomaterial 104 can be injected into the LAA through the injection channel of the third catheter body. The photocurable biomaterial can be flowable and conform to the internal anatomy of the LAA prior to crosslinking.

Subsequently, the third catheter body can be removed from the primary opening of the second catheter. A delivery system sized to be received within the primary opening of, and selectively moveable relative to, the second catheter body can then be inserted into the primary opening of the second catheter body. The delivery system can comprise: a delivery catheter body extending between a proximal end and a distal end, a handle coupled to the proximal end of the delivery catheter body, and an occlusion device operatively coupled to the handle and coupled to the distal end of the delivery catheter body, wherein the occlusion device comprises an occluder portion and an anchor portion operably coupled to the occluder portion. As shown in FIG. 13C, the delivery system 122 can then be advanced within the primary opening of the second catheter body such that the occlusion device 102 is passed through the primary opening of the second catheter body to a position distal of the second balloon 120. The occlusion device 102 can then be deployed within the ostium of the LAA 108 such that the anchor portion 112 extends into the internal volume of the LAA 106.

Subsequently, the photocurable biomaterial can be irradiated with actinic radiation, thereby inducing crosslinking of the photocurable biomaterial in situ in the LAA to form the IPN or sIPN that fills and occupies the internal volume of the LAA. The occlusion device can be retained within the ostium of the LAA until irradiation and photocrosslinking have occurred and the photocurable biomaterial has solidified to form the IPN or sIPN. At this point, delivery catheter assembly can be withdrawn, leaving the occlusion device in place. As shown in FIG. 13D, because of the compliant nature of the photocurable biomaterial, the resulting IPN or sIPN (116) can be interpenetrated by both the anchor portion (112) of the occlusion device and trabeculae present in the LAA (not shown for simplicity). In this way, the IPN or sIPN ensures that the occlusion device 102 is retained within the ostium of the LAA 108. Further, the IPN or sIPN can function as an occlusive body, filling and occupying the internal volume of the LAA without adversely impacting cardiac function. In addition, the occlusion device can isolate the IPN or sIPN from blood present in the left atrium, provide a scaffold for endothelialization, of a combination thereof.

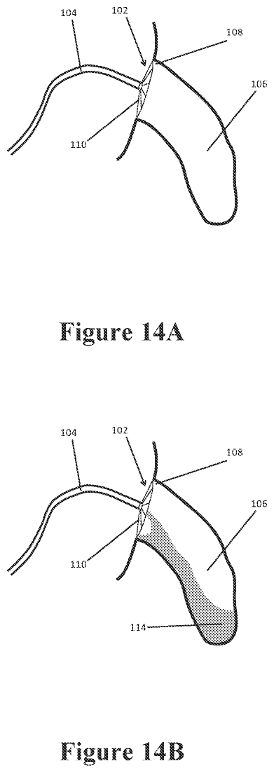

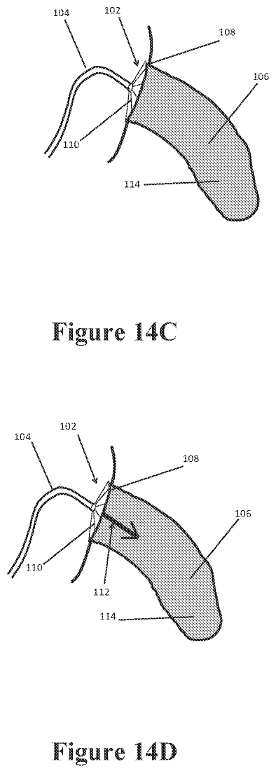

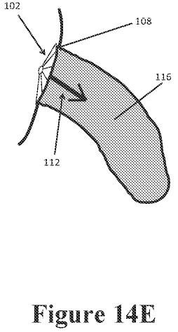

An alternative method for occluding the LAA of a patient is illustrated in FIGS. 14A-14E. These methods can comprise advancing a delivery system percutaneously through the patient's vasculature to reach the patient's right atrium. The delivery system can comprise a delivery catheter, wherein the delivery catheter comprises: a delivery catheter body extending between a proximal end and a distal end, the catheter body comprising a wall structure that defines at least one injection channel extending from the proximal end to the distal end and terminating in an outlet opening; a handle coupled to the proximal end of the delivery catheter body, and an occlusion device operatively coupled to the handle and coupled to the distal end of the delivery catheter body, wherein the occlusion device comprises an occluder portion comprising a proximal end and a distal end, the proximal end coupled to a hub having an injection lumen passing axially therethrough, wherein the outlet opening of the at least one injection channel is fluidly connected to the injection lumen of the occlusion device. Next, the delivery system can be advanced through an opening in the interatrial septum to reach the patient's left atrium. As shown in FIG. 14A, the occlusion device 102 can then be deployed within the ostium of the LAA 108 using the delivery catheter 104. When deployed, the occluder portion 110 of the occlusion device 102 can substantially isolate the internal volume of the LAA (106) from the left atrium. Next, as shown in FIGS. 14B and 14C, a photocurable biomaterial 114 can be injected into the LAA through the injection channel of the delivery catheter body and the injection lumen of the occlusion device 102. The photocurable biomaterial can be flowable, allowing it to comply with the irregular shape of the interior of the LAA. The photocurable biomaterial can then be irradiated with actinic radiation, thereby inducing crosslinking of the photocurable biomaterial in situ in the LAA to form the IPN or sIPN that fills and occupies the internal volume of the LAA.

As shown in FIG. 14D, an anchoring portion can then be advanced through the injection channel of the delivery catheter body and the injection lumen and coupling the anchoring portion 114 to the occlusion device 102. When the anchoring portion 112 is coupled to the occlusion device 102, the anchor portion 102 extends into the internal volume of the LAA 106. At this point, delivery catheter 104 can be withdrawn, leaving the occlusion device in place. As shown in FIG. 14E, because of the compliant nature of the photocurable biomaterial, the resulting IPN or sIPN (116) can be interpenetrated by both the anchor portion (112) of the occlusion device and trabeculae present in the LAA (not shown for simplicity). In this way, the IPN or sIPN ensures that the occlusion device is retained within the ostium of the LAA 108. Further, the IPN or sIPN can function as an occlusive body, filling and occupying the internal volume of the LAA without adversely impacting cardiac function. In addition, the occlusion device can isolate the IPN or sIPN from blood present in the left atrium, provide a scaffold for endothelialization, of a combination thereof.

In practicing the methods described herein, the delivery catheters/systems can be inserted into the vasculature of the patient (e.g., into the femoral vein), and advanced through the patient's vasculature, such that they reach the patient's left atrium. The LAA may be accessed through any of a variety of pathways as will be apparent to those of skill in the art. Trans-septal access can be achieved by introducing the delivery catheter/system into, for example, the femoral or jugular vein, and transluminally advancing the catheter into the right atrium. Radiographic imaging (e.g., single or biplanar fluoroscopy, sonographic imaging, or combinations thereof) can be used to image the delivery catheter during the procedure and guide the distal end of the catheter to the desired site. As a result, in some cases, at least a portion of the delivery catheter can be formed to be at least partially radiopaque.

Once in the right atrium, a long hollow needle with a preformed curve and a sharpened distal tip can be advanced through the delivery catheter/sheath, and forcibly inserted through the fossa ovalis. A radiopaque contrast media can be injected through the needle to allow visualization and ensure placement of the needle in the left atrium, as opposed to being in the pericardial space, aorta, or other undesired location. Once the position of the needle in the left atrium is confirmed, the delivery catheter/sheath can be advanced over the needle through the septum and into the left atrium. Alternative approaches to the LAA are known in the art, and can include venous transatrial approaches such as transvascular advancement through the aorta and the mitral valve.

If desired, fluid (e.g., blood) present in the LAA can be removed following sealing of the LAA (e.g., with the occlusion device) but prior to injection of the photocurable biomaterial. Optionally, the volume of blood removed from the LAA of the patient can be measured, and used to determine an appropriate amount of photocurable biomaterial to be injected into the LAA of the patient. In another embodiment, diagnostic imaging and image analysis can be utilized to determine an appropriate amount of photocurable biomaterial to be injected into the LAA of the patient.

In some embodiments, the total volume of photocurable biomaterial injected ranges from about 2 mL to about 15 mL (e.g., from 2 mL to about 10 mL, or from about 5 mL to about 15 mL). The photocurable biomaterial can be injected via one or more lumens, for example, using a syringe, inflator, or other device fluidly connected to the one or more lumens. Upon injection into the LAA and irradiation, the photocurable biomaterial photocrosslinks and increases in viscosity to form an IPN or sIPN.

In some embodiments, during the procedure described above, the patient can be positioned in a posture which is effective to facilitate occlusion of the LAA. For example, the patient can be positioned at an angle relative to the ground which is effective to facilitate injection of the photocurable biomaterial into the LAA of the patient. By positioning the patient at an angle (e.g., approximately a 30° to 40° angle relative to horizontal), gravity can assist the flow of the photocurable biomaterial into the LAA, facilitating complete occlusion of the LAA.

In general, the methods described herein are performed percutaneously, for example using a delivery catheter assembly as discussed above. Alternatively, the photocurable biomaterial and occlusion device can be introduced intraoperatively during an invasive procedure, or ancillary to another procedure which gives access to the LAA.

The methods described herein can be used to occlude the LAA, thus decreasing the risk of thromboembolic events associated with AF.

In some cases, the patient treated using the methods described herein exhibits AF. In patients with non-rheumatic AF, the risk of stroke can be estimated by calculating the patient's CHA₂DS₂-VASc score. A high CHA₂DS₂-VASc score corresponds to a greater risk of stroke, while a low CHA₂DS₂-VASc score corresponds to a lower risk of stroke. In some embodiments, the patient treated using the methods described has a CHA₂DS₂-VASc score of 2 or more.

In certain embodiments, the patient is contraindicated for anticoagulation therapy. For example, the patient can have an allergy to one or more common anticoagulants (e.g. warfarin), can express a preference to not be treated with anticoagulants, can be taking another medication that interacts unfavorably with an anticoagulant, or can be at risk for hemorrhage.

### EXAMPLES

### Example 1: Synthesis and Evaluation of an Example Fluid Biomaterial

### Preparation of Hydrogel Precursor Molecules

Tetra-functional PEG-thiol (PEG4SH) (82.7% activity) was purchased from Sunbio (Anyang City, South Korea), and used for hydrogel formation without further purification or modification.

Dextran from Leuconostoc mesenteroides (average MW=15,000-20,000 Da), divinyl sulfone (DVS; 97%, MW=118.15 Da), and 3-mercaptopropionic acid (MW=106.14 Da) were purchased from Sigma-Aldrich (St. Louis, MO). The synthesis of dextran vinyl sulfone (DextranVS) containing an ethyl spacer was performed using N,N0-dicyclohexyl-carbodiimide (DCC, Fisher Scientific) and 4-(dimethylamino)pyridinium 4-toluenesulfonate (DPTS) as catalysts. DPTS was prepared using methods known in the art. Briefly, 5.0 g of p-TSA monohydrate was dissolved in 100 ml THF. 4-(Dimethylamino)-pyridine (DMAP, 99%) (Sigma-Aldrich, St. Louis, MO) at one molar equivalent top-TSA was added to this mixture. The mixture was subsequently filtered to isolate a precipitate which was further dissolved in dichloromethane (DCM, Fisher Scientific) and recrystallized using rotary vacuum evaporator.

Dextran vinyl sulfone ester synthesis was performed by adding 2.5 or 5.0 g DVS in 90 mL of inert nitrogen saturated DMSO, followed by dropwise addition of 3-MPA to it under continuous stirring. The reaction was continued for 4 hours in the dark. Dextran was dissolved in 30 mL DMSO, and a solution of DCC and p-TSA in 30 ml DMSO was added dropwise. The reaction mixture was stirred until a clear solution was obtained. Finally, the mixture was added to DVS/MPA solution in the dark, and reaction was allowed to proceed for 24 hours at room temperature.

After the completion of reaction, N,N-dicyclohexylurea (DCU) salt was filtered using a vacuum filter and the product was recovered by precipitation in 1000 mL of ice cold 100% ethanol. The precipitate was separated from residual ethanol through centrifugation at 3000 rpm for 15 min., followed by vacuum drying. The precipitate was re-dissolved in at least 100 mL of de-ionized water (pH adjusted to 7.8) and vortexed to obtain a clear solution. Finally, un-reacted polymer was removed via ultra-filtration using an Amicon filter (MWCO=10,000 Da, Millipore). The resulting viscous product was lyophilized to remove water. Vinyl sulfone substitution was confirmed and degree of substitution (DS) was determined via NMR spectroscopy.

### Formation of PEG-Dextran Hydrogels

Controlled masses of PEG and dextran vinyl sulfone were mixed with a controlled volume of TEA buffer. Two different types of dextran vinyl sulfone (DS 5 and DS10) were examined. Samples were made with varying concentrations of hydrogel in the buffer, measured in terms of wt. %/vol. Samples ranging from 10%-40% wt./vol. were evaluated. The PEG and dextran components were mixed in a 1:1 stoichiometric ratio.

### Characterization of PEG-Dextran Hydrogels

The materials properties of the PEG-Dextran hydrogels, as well as solutions of the hydrogel precursor molecules were evaluated.

### Measurement of Density and Viscosity

A controlled volume (500 μL) of each sample was collected, and weighed. Knowing the mass and volume, density was calculated. The densities measured for solutions of hydrogel precursor molecules are included in Table 1.

TABLE 1
Density measurements for solutions of hydrogel precursor molecules.
Table 1: Density (g/cm³) of Materials

| | | | | |
|---|---|---|---|---|
| Concentration | 10% | 20% | 30% | 40% |

| | | | | | |
|---|---|---|---|---|---|
| Material | PEG | 1.080 | 1.202 | 1.295 | 1.393 |
| | Dextran DS 5 | 1.120 | 1.191 | 1.305 | 1.395 |
| | Dextran DS 10 | 1.107 | 1.193 | 1.306 | 1.399 |

Samples of hydrogel components were prepared as described previously, and heated to 37° C. in a water bath. Solutions of the hydrogel precursor molecules were also measured. Kinematic viscosities were measured using size 75 and size 150 Canon Manning Semi-Micro glass capillary viscometers. Once kinematic viscosity was measured, dynamic viscosity was calculated using the following relation:

$$v = \frac{μ}{ρ}$$

Where ν is kinematic viscosity, μ is dynamic viscosity, and ρ is density of the measured material. For each sample, viscosity was measured 12 times to ensure accuracy. The dynamic and kinematic viscosities of solutions of hydrogel precursor molecules at different concentrations are included in Table 2. The standard deviation of all measurements was found to be relatively small (<1.4% for all hydrogels measured).

TABLE 2
Dynamic and kinematic viscosities of solutions
of hydrogel precursor molecules.

| | | | | |
|---|---|---|---|---|
| Concentration | 10% | 20% | 30% | 40% |

Dynamic Viscosity (cP) of Materials

| | | | | | |
|---|---|---|---|---|---|
| Material | PEG | 2.646 | 7.773 | 24.729 | 32.810 |
| | Dextran DS 5 | 1.789 | 3.859 | 7.371 | 11.871 |
| | Dextran DS 10 | 1.607 | 4.075 | 6.478 | 16.335 |

Kinematic Viscosity (cStr) of Materials

| | | | | | |
|---|---|---|---|---|---|
| Material | PEG | 2.452 | 6.467 | 19.100 | 23.556 |
| | Dextran DS 5 | 1.598 | 3.239 | 5.649 | 8.510 |
| | Dextran DS 10 | 1.451 | 3.416 | 4.960 | 11.673 |

### Measurement of Degradation Rate

Samples of PEG and Dextran were mixed in a 1:1 ratio and allowed to solidify. In this study, 150 μL of each component was used. Samples were allowed to sit for two hours to allow complete solidification. To simulate human body conditions, samples were then submerged in a 0.01% PBS buffer (PH 7.4), and rotated in a 37° C. incubator. Samples were weighed at specified time intervals to gauge what percentage of material remained.

The results of the degradation trials are included in Table 3. After 90 days anywhere between 40-63% of hydrogel (by mass) had degraded. At 120 days, anywhere between 44-95% of hydrogel by mass had degraded.

TABLE 3
Degradation results for PEG-dextran hydrogels.
Mass/Original Mass (Swelling Ratio)

| | DS 5 | DS 10 | DS 5 | DS 10 | DS 5 | DS 10 |
|---|---|---|---|---|---|---|
| Day | 20% | 20% | 30% | 30% | 40% | 40% |
| 0 | 1.00 | 1.00 | 1.00 | 1.00 | 1.00 | 1.00 |
| 1 | 1.52 | 1.74 | 3.13 | 3.30 | 3.32 | 3.73 |
| 2 | 2.55 | 2.49 | 3.03 | 3.37 | 3.46 | 5.89 |
| 8 | 2.63 | 2.68 | 3.43 | 3.09 | 3.70 | 4.16 |
| 15 | 2.28 | 2.52 | 2.62 | 2.85 | 2.86 | 3.41 |
| 23 | 2.02 | 2.31 | 2.15 | 2.78 | 2.90 | 3.04 |
| 29 | 2.44 | 2.07 | 2.15 | 2.66 | 2.85 | 3.44 |
| 42 | 2.04 | 1.73 | 1.88 | 2.28 | 2.32 | 2.98 |
| 54 | 2.02 | 1.71 | 1.61 | 2.17 | 2.31 | 2.61 |
| 64 | 2.01 | 2.11 | 1.87 | 2.33 | 2.62 | 2.82 |
| 79 | 1.95 | 1.95 | 1.73 | 1.95 | 2.20 | 2.44 |
| 86 | 1.66 | 1.76 | 1.57 | 2.08 | 1.99 | 2.40 |
| 92 | 1.59 | 1.74 | 1.28 | 1.79 | 1.94 | 2.29 |
| 99 | 1.52 | 1.60 | 1.28 | 1.93 | 1.88 | 2.51 |
| 106 | 1.62 | 1.59 | 1.30 | 1.96 | 2.10 | 2.48 |
| 113 | 1.51 | 1.59 | 1.26 | 1.90 | 1.95 | 2.40 |
| 120 | 1.47 | 1.71 | 1.29 | 1.79 | 1.94 | 2.43 |
| 127 | 1.46 | 1.59 | | 1.71 | 1.92 | 2.35 |

% Remaining

| | DS 5 | DS 10 | DS 5 | DS 10 | DS 5 | DS 10 |
|---|---|---|---|---|---|---|
| Day | 20% | 20% | 30% | 30% | 40% | 40% |
| 0 | | | | | | |
| 1 | | | | | | |
| 2 | | | | 1.00 | | 1.00 |
| 8 | 1.00 | 1.00 | 1.00 | 0.92 | 1.00 | 0.71 |
| 15 | 0.87 | 0.94 | 0.76 | 0.85 | 0.77 | 0.58 |
| 23 | 0.77 | 0.86 | 0.63 | 0.83 | 0.78 | 0.52 |
| 29 | 0.93 | 0.77 | 0.63 | 0.79 | 0.77 | 0.58 |
| 42 | 0.78 | 0.64 | 0.55 | 0.68 | 0.63 | 0.51 |
| 54 | 0.77 | 0.64 | 0.47 | 0.64 | 0.62 | 0.44 |
| 64 | 0.77 | 0.79 | 0.55 | 0.69 | 0.71 | 0.48 |
| 79 | 0.74 | 0.73 | 0.50 | 0.58 | 0.59 | 0.41 |
| 86 | 0.63 | 0.66 | 0.46 | 0.62 | 0.54 | 0.41 |
| 92 | 0.61 | 0.65 | 0.37 | 0.53 | 0.52 | 0.39 |
| 99 | 0.58 | 0.60 | 0.37 | 0.57 | 0.51 | 0.43 |
| 106 | 0.62 | 0.59 | 0.38 | 0.58 | 0.57 | 0.42 |
| 113 | 0.57 | 0.59 | 0.37 | 0.56 | 0.53 | 0.41 |
| 120 | 0.56 | 0.64 | 0.38 | 0.53 | 0.52 | 0.41 |
| 127 | 0.56 | 0.59 | 0.00 | 0.51 | 0.52 | 0.40 |

% Degraded

| | DS 5 | DS 10 | DS 5 | DS 10 | DS 5 | DS 10 |
|---|---|---|---|---|---|---|
| Day | 20% | 20% | 30% | 30% | 40% | 40% |
| 0 | | | | | | |
| 1 | | | | | | |
| 2 | | | | 0.00 | | 0.00 |
| 8 | 0.00 | 0.00 | 0.00 | 0.08 | 0.00 | 0.29 |
| 15 | 0.13 | 0.06 | 0.24 | 0.15 | 0.23 | 0.42 |
| 23 | 0.23 | 0.14 | 0.37 | 0.17 | 0.22 | 0.48 |
| 29 | 0.07 | 0.23 | 0.37 | 0.21 | 0.23 | 0.42 |
| 42 | 0.22 | 0.36 | 0.45 | 0.32 | 0.37 | 0.49 |
| 54 | 0.23 | 0.36 | 0.53 | 0.36 | 0.38 | 0.56 |
| 64 | 0.23 | 0.21 | 0.45 | 0.31 | 0.29 | 0.52 |
| 79 | 0.26 | 0.27 | 0.50 | 0.42 | 0.41 | 0.59 |
| 86 | 0.37 | 0.34 | 0.54 | 0.38 | 0.46 | 0.59 |
| 92 | 0.39 | 0.35 | 0.63 | 0.47 | 0.48 | 0.61 |
| 99 | 0.42 | 0.40 | 0.63 | 0.43 | 0.49 | 0.57 |
| 106 | 0.38 | 0.41 | 0.62 | 0.42 | 0.43 | 0.58 |
| 113 | 0.43 | 0.41 | 0.63 | 0.44 | 0.47 | 0.59 |
| 120 | 0.44 | 0.36 | 0.62 | 0.47 | 0.48 | 0.59 |
| 127 | 0.44 | 0.41 | 1.00 | 0.49 | 0.48 | 0.60 |

### Measurement of Equilibrium Swelling Ratio

Hydrogel samples were obtained and massed. The hydrogel samples were then incubated in PBS buffer (10 mM phosphate buffered saline, e.g., P3813-powder from Sigma yields a buffer of 0.01 M phosphate, 0.0027 M potassium chloride and 0.138 M sodium chloride, pH 7.4). Upon incubation, the hydrogel samples swelled, and increased mass. Every ˜168 hours (7 days), the sample was removed from buffer, and massed. The equilibrium swelling ratio, defined as:

$$equilibrium swelling ratio = \frac{{w}_{t}}{{w}_{o}}$$

where wₜ is the maximum swollen weight, and wo is the unswollen weight of hydrogel was determined for each hydrogel sample. Equilibrium swelling ratio was typically observe 48-72 hours after submerging samples in PBS buffer. The equilibrium swelling ratios of each hydrogel are included in Table 4 below.

TABLE 4
Equilibrium swelling ratios of PEG-Dextran hydrogels.

| Dextran | Concentration of Hydrogel Components | | |
|---|---|---|---|
| Component | 20% | 30% | 40% |
| Dextran DS 5 | 2.6 | 3.4 | 3.7 |
| Dextran DS 10 | 2.6 | 3.3 | 5.8 |

### Measurement of Cure Time

The cure time of PEG-dextran hydrogels was evaluated using a tipping vial methods. PEG and Dextran DS 5 suspensions were prepared, as described above, at different concentrations by mixing either PEG or dextran material with TEA buffer. Suspensions were mixed to a specific concentration. PEG and dextran suspensions of equal concentrations were then mixed in a 1:1 ratio in a sealed vial. The vial was shaken with an ultrasonic shaker to ensure complete mixing. Once mixing was complete, a timer was started. The vial was continually tipped or flipped. When mixed components stop moving upon actuation of the vial, the hydrogel is considered gelled, and the timer was stopped.

For each concentration, 5 solidification trials were performed. The average cure time (in seconds) for each PEG-Dextran DS 5 hydrogel measured is included in the Table 5 below, along with the standard deviation for each cure time.

TABLE 5
Cure time of of PEG-Dextran DS 5 hydrogels.

| Hydrogel | Mean Solidification | Standard Deviation |
|---|---|---|
| Concentration | Time (seconds) | (seconds) |
| 20% | 99 | 2.94 |
| 30% | 84 | 5.48 |
| 40% | 58.25 | 2.22 |

Concentration of the hydrogel sample was found to be inversely proportional to the cure time. In addition, a statistically significant correlation between concentration and cure time was observed. This was believed to be due to higher concentrations exhibiting a faster rate of crosslinking. The standard deviations for cure time were also relatively small (<6.6% of mean cure time for all samples), indicating solidification times are relatively consistent from sample to sample.

### Measurement of Volumetric Swelling Ratio

During the studies described above, maximum swelling was typically observed after 1-2 days submerged in buffer. Generally, higher concentrations and DS numbers resulted in hydrogels that exhibited greater swelling in terms of mass. To better understand the relationship between material configurations and volumetric swelling, the volumetric swelling of various hydrogel samples was evaluated.

Samples from the cure time trials described above were allowed to sit for 2 hours to solidify completely. For each aqueous solution concentration, 5 separate hydrogel samples were used. Samples were then submerged in PBS buffer, and allowed to swell. Every 24 hours, samples were removed from the buffer and dried, and the sample's volume was measured using a graduated cylinder. Volumetric swelling ratio is defined as:

$$volumetric swelling ratio = \frac{{V}_{t}}{{V}_{o}}$$

where Vₜ is the swollen volume of the hydrogel sample and Vₒ is the unswollen volume of the hydrogel sample. The mean volumetric swelling ratios of the PEG and Dextran DS 5 samples at 24 hours, 48 hours, and 72 hours are plotted in FIG. 5.

In general, aqueous solutions with higher concentrations resulted in hydrogels that exhibited higher volumetric swelling. Most of this swelling was found to occur within the first 24 hours of incubation in buffer.

### Hydrogel Anchoring Analysis

To be suitable for occlusion of the LAA, the hydrogel should remain anchored in the LAA following transport and solidification in the LAA during occlusion. The swelling of hydrogel samples over time (determined above) was used to estimate the interface pressures exerted by various hydrogel samples. The anchoring force resulting from these estimated interface pressures could then be estimated.

The degradation results described above evaluated the hydrogel composition over time. From these trials, it was possible to extrapolate a projected equilibrium swelling ratio (based on mass measurements) for hydrogel samples at 180 days (the estimated time period needed for complete endothelial tissue overgrowth over the opening of the LAA). While volumetric swelling ratios would provide a more accurate projection, long-term volumetric degradation data was not available for analysis. As a result, it was assumed that volumetric degradation was approximately proportional to mass-based degradation.

For purposes of this estimate, it was assumed that the hydrogel undergoes isotropic swelling, and that the swelling in terms of mass corresponded to a dimensionally similar volumetric swelling. Change in hydrogel plug radius can be predicted based on these swelling ratios with the following relationship:

$$Radius Multiple , {R}_{m} = \frac{{R}_{swollen}}{{R}_{initial}} = {Q}_{m}^{1 / 3}$$

Tables 6 includes the projected Qₘ and Rₘ at 180 days.

TABLE 6
Projected swelling ratio and material radius at 180 days.

| Hydrogel Sample | w/wₒ at 180 Days | R/Rₘ at 180 Days |
|---|---|---|
| PEG-Dextran DS 5 20% | 1.449 | 1.131 |
| PEG-Dextran DS 5 30% | 1.117 | 1.037 |
| PEG-Dextran DS 5 40% | 1.767 | 1.208 |
| PEG-Dextran DS 10 20% | 1.509 | 1.146 |
| PEG-Dextran DS 10 30% | 1.686 | 1.190 |
| PEG-Dextran DS 10 40% | 2.165 | 1.293 |

To analyze anchoring force, the interface pressure was then estimated. The interface pressure between the hydrogel plug and LAA tissue was defined with the following equation.

$$Interface Pressure , {p}_{i} = \frac{{δ}_{r}}{\frac{R}{{E}_{o}} ( \frac{{r}_{o}^{2} + {R}^{2}}{{r}_{o}^{2} - {R}^{2}} + {v}_{o} ) + \frac{R}{{E}_{i}} ( 1 - {v}_{i} )}$$

where δᵣ is the change in hydrogel radius due to swelling; R is the initial radius of the hydrogel plug; rₒ is the outer diameter of LAA tissue; νₒ and νᵢ are Poisson's ratio for tissue and the hydrogel, respectively, and Eₒ and Eₗ are the elastic modulus of the tissue and hydrogel, respectively. Once estimated, the interface pressure was used to calculate the anchoring force of the hydrogel using the equation below:

F=πdLₕρᵢμ_{f}

where pᵢ is the interface pressure described previously; d is the diameter of the hydrogel (equal to 2R); Lₕ is the length of contact surface between tissue and hydrogel; and μ_{f} is the coefficient of friction between LAA tissue and the hydrogel. The values used for the variables in the anchoring force analysis are included in Table 7 below.

TABLE 7
Values used for the variables in the anchoring force analysis.

| Variable | Description | Value | |
|---|---|---|---|
| δᵣ | Change in hydrogel radius | Varies by sample | |
| R | Initial radius of hydrogel | 10 | mm |
| r | Initial radius of hydrogel and tissue | 11 | mm |
| E₀ | Elastic modulus of tissue | 40.8 | kPa |
| E₁ | Elastic modulus of hydrogel | 14 | kPa |
| ν₀ | Poisson Ratio of tissue | 0.45 | |
| ν₁ | Poisson Ratio of hydrogel | 0.5 | |
| d | Diameter of hydrogel | 20 | mm |
| Lₕ | Length of hydrogel | 30 | mm |

| | | |
|---|---|---|
| μ_{f} | Coefficient of friction | 0.332 |

The anchoring force for each hydrogel sample was compared to the estimated weight of the hydrogel plug. Samples were determined to exhibit adequate anchoring force for use in occlusion of the LAA if the hydrogel plug exhibited an estimated anchoring force at least as large as the weight of the hydrogel plug. Experimentally measured values for density were used to estimate the weight of each hydrogel plug. Table 8 below shows the estimated weight and anchoring force of each hydrogel plug, based upon projected swelling ratios at day 180 and the equations above.

TABLE 8
Estimated weight and anchoring force for plugs
formed from various PEG-Dextran hydrogels.

| | Hydrogel | Plug | Anchoring | Anchoring |
|---|---|---|---|---|
| | Density | Weight | Force | Force/Plug |
| Hydrogel Sample | (g/cm³) | (N) | (N) | Weight |
| PEG-Dextran DS 5 20% | 1.195 | 0.221 | 0.277 | 1.253 |
| PEG-Dextran DS 5 30% | 1.300 | 0.240 | 0.078 | 0.325 |
| PEG-Dextran DS 5 40% | 1.395 | 0.258 | 0.444 | 1.721 |
| PEG-Dextran DS 10 20% | 1.195 | 0.221 | 0.310 | 1.403 |
| PEG-Dextran DS 10 30% | 1.300 | 0.240 | 0.403 | 1.679 |
| PEG-Dextran DS 10 40% | 1.395 | 0.258 | 0.631 | 2.445 |

Based on the results above, all samples other than the DS 5 30%, exhibited estimated anchoring forces at least as large as the weight of the hydrogel plug with a safety factor of 1.25 or higher. The low value for the DS 5 30% sample was likely due to anomalous degradation data. A more reasonable range for anchoring force would be 0.277-0.444N (in between DS 5 20% and DS 5 40% samples), which would suggest that the DS 5 30% material would also exhibit adequate anchoring force for use in occlusion of the LAA.

It is worth noting that this analysis represents the most conservative anchoring scenario. Additional anchoring forces due to blood pressure and geometric anomalies (e.g., irregular trabeculae or multiple lobes in the LAA) were not considered. Blood pressure would likely only act on the outer surface of the hydrogel (the surface facing the LA), providing additional anchoring force. Irregular trabeculae and geometric asymmetries in the LAA would also cause additional anchoring, but these were not quantified due to unpredictable geometry in the LAA. Both of these would add additional anchoring, and provide further evidence that this hydrogel is suitable for application in occluding the LAA.

The devices and methods of the appended claims are not limited in scope by the specific devices, systems, kits, and methods described herein, which are intended as illustrations of a few aspects of the claims. Any devices, systems, and methods that are functionally equivalent are intended to fall within the scope of the claims. Various modifications of the devices, systems, kits, and methods in addition to those shown and described herein are intended to fall within the scope of the appended claims. Further, while only certain representative devices, systems, kits, and method steps disclosed herein are specifically described, other combinations of the devices, systems, kits, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method of occluding a left atrial appendage (LAA) of a patient comprising:
injecting a photocurable biomaterial into the LAA of the patient; and
irradiating the photocurable biomaterial with actinic radiation, thereby inducing crosslinking of the photocurable biomaterial in situ in the LAA to form an interpenetrating network (IPN) or semi-interpenetrating network (sIPN) comprising a first network polymer and a second network polymer;
wherein the first network polymer comprises a hydrophilic polymer; and
wherein the second network polymer comprises a silicone rubber.

2. The method of claim 1, wherein the hydrophilic polymer is selected from the group consisting of polyethers, polyacrylates, polyesters, polyanhydrides, polyols, polypeptides, polyvinyl alcohols, proteins, polysaccharides, gelatins, elastins, collagens, celluloses, methylcelluloses, hyaluronic acid, dextrans, alginates, copolymers thereof, and derivatives thereof.

3. The method of any of claims 1-2, wherein the hydrophilic polymer comprises a non-biodegradable polymer.

4. The method of any of claims 1-3, wherein the hydrophilic polymer comprises a synthetic polymer.

5. The method of any of claims 1-4, wherein the hydrophilic polymer comprises a hydrophilic polyacrylate, such as poly(hydroxyethyl)methacrylate or a copolymer thereof.

6. The method of any of claims 1-4, wherein the hydrophilic polymer comprises a hydrophilic urethane acrylate.

7. The method of any of claims 1-6, wherein the photocurable biomaterial comprises a bifunctional epoxy siloxane monomer.

8. The method of claim 7, wherein the bifunctional epoxy siloxane monomer comprises one or more hydrophilic functional groups, such as one or more hydroxy groups, one or more carboxylic acid groups, or a combination thereof.

9. The method of any of claims 7-8, wherein the photocurable biomaterial further comprises one or more additional epoxy monomers.

10. The method of claim 9, wherein the one or more additional epoxy monomers comprise one or more hydrophilic functional groups, such as one or more hydroxy groups, one or more carboxylic acid groups, or a combination thereof.

11. The method of any of claims 1-10, wherein the one or more additional epoxy monomers comprise one or more polyfunctional epoxy siloxane monomers.

12. The method of claim 11, wherein the one or more polyfunctional epoxy siloxane monomers comprise at least three epoxy groups.

13. The method of any of claims 11-12, wherein the one or more polyfunctional epoxy siloxane monomers and the bifunctional epoxy siloxane monomer are present in the photocurable biomaterial at a molar ratio of from 0.01:100 to 15:100, such as from 0.05:100 to 10:100 or from 0.1:100 to 5:100.

14. The method of any of claims 1-13, wherein the silicone rubber comprises a hydrophilic silicone rubber.

15. The method of any of claims 1-14, wherein the photocurable biomaterial further comprises one or more (meth)acrylate monomers.

16. The method of any of claims 1-15, wherein the first network polymer and the second network polymer are co-continuous.

17. The method of any of claims 1-16, wherein crosslinking of the photocurable biomaterial in situ in the LAA to form an interpenetrating network (IPN).

18. The method of any of claims 1-17, wherein the first network polymer comprises at least 30% by weight of the IPN or sIPN, based on the total weight of all network polymers forming the IPN or sIPN.

19. The method of any of claims 1-18, wherein the first network polymer comprises from 30% by weight to 80% by weight of the IPN or sIPN, based on the total weight of all network polymers forming the IPN or sIPN.

20. The method of any of claims 1-19, wherein the photocurable biomaterial has a viscosity of from 1 cP to 10,000 cP at 25° C.

21. The method of any of claims 1-20, wherein the IPN or sIPN exhibits a viscosity of at least 500,000 at body temperature (e.g., at 37° C.).

22. The method of any of claims 1-21, wherein irradiating the photocurable biomaterial with actinic radiation comprises delivering at least 5 J/cm³ of energy to the photocurable biomaterial

23. The method of any of claims 1-22, wherein the IPN or sIPN exhibits an equilibrium swelling ratio of from greater than 0 to about 10, such as from greater than 0 to about 8.

24. The method of any of claims 1-23, wherein the IPN or sIPN exhibits a volumetric swelling ratio of from greater than 0 to about 15, such as from greater than 0 to about 10, or from about 2 to about 8.

25. The method of any of claims 1-24, wherein the IPN or sIPN has an elastic modulus of from about 5 kPa to about 20 kPa, such as from about 8 kPa to about 12 kPa.

26. The method of any of claims 1-25, wherein the IPN or sIPN further comprises a bioactive agent dispersed therein.

27. The method of claim 26, wherein the bioactive agent comprises a silencing agent, such as an apoptotic agent.

28. The method of any of claims 26-27, wherein the bioactive agent comprises a contrast agent.

29. The method of any of claims 1-28, wherein the patient exhibits atrial fibrillation.

30. The method of any of claims 1-29, wherein the LAA is trabeculated.

31. The method of claim 30, wherein the photocurable biomaterial conforms to the internal anatomy of the LAA prior to crosslinking, such that the resulting IPN or sIPN is entrained within trabeculae present in the LAA.

32. The method of any of claims 1-31, wherein the patient has a CHA₂DS₂-VASc score of 2 or more.

33. The method of any of claims 1-32, wherein the patient is contraindicated for anticoagulation therapy.

34. The method of any of claims 1-33, wherein the LAA extends from a left atrium of the patient's heart and has an internal volume and an ostium at its juncture with the left atrium; and
wherein the method comprises:
positioning an occlusion device within the ostium of the LAA,
wherein the occlusion device comprises an occluder portion comprising a proximal end and a distal end, the proximal end coupled to a hub having an injection lumen passing axially therethrough; and an anchor portion operably coupled to the occluder portion; and
wherein when the occlusion device is positioned within the ostium of the LAA, the anchor portion extends into the internal volume of the LAA;
injecting the photocurable biomaterial into the LAA of the patient through the injection lumen, wherein the photocurable biomaterial is flowable and conforms to the internal anatomy of the LAA prior to crosslinking;
irradiating the photocurable biomaterial with actinic radiation, thereby inducing crosslinking of the photocurable biomaterial in situ in the LAA to form the IPN or sIPN that fills and occupies the internal volume of the LAA; and
retaining the occlusion device within the ostium of the LAA until the IPN or sIPN has formed.

35. The method of claim 34, wherein the occluder portion is configured to move between an occluder-deployed state and an occluder-nondeployed state, and
wherein the anchor portion is configured to move between an anchor-deployed state and an anchor-nondeployed state.

36. The method of any of claims 34-35, wherein the anchor portion comprises a plurality of anchor segments, wherein each of the plurality of anchor segments extend distally beyond the occluder portion when the occlude portion is in the occlude-deployed state and the anchor portion is in the anchor-deployed state.

37. The method of claim 36, wherein each of the anchor segments comprises a loop portion, a helical portion, a fin portion, a barb portion, or any combination thereof.

38. The method of any of claims 34-37, wherein the anchor portion is coupled to the occluder portion by way of the hub.

39. The method of any of claims 34-38, wherein the occluder portion comprises a tissue growth member extending between the proximal end and the distal end of the occluder portion.

40. The method of claim 39, wherein the tissue growth member comprises a layer formed from an expanded polytetrafluoroethylene (ePTFE).

41. The method of any of claims 34-40, wherein the hub further comprises a second lumen passing axially therethrough, wherein the second lumen is fluidly isolated from the injection lumen.

42. The method of claim 41, wherein the injection channel terminates distally at an injection outlet and the second lumen terminates distally at a fluid inlet.

43. The method of claim 42, wherein the injection outlet is separated from and distal to the fluid inlet.

44. The method of any of claims 34-43, wherein the occlusion device is positioned within the ostium of the LAA using a delivery system, wherein the delivery system comprises:
a delivery catheter, wherein the delivery catheter comprises:
a delivery catheter body extending between a proximal end and a distal end, the catheter body comprising a wall structure that defines at least one injection channel extending from the proximal end to the distal end and terminating in an outlet opening;
a handle coupled to the proximal end of the delivery catheter body, and
the occlusion device operatively coupled to the handle and coupled to the distal end of the delivery catheter body,
wherein the outlet opening of the at least one injection channel is fluidly connected to the injection lumen of the occlusion device.

45. The method of claim 44, wherein the delivery system further comprises a sheath having a proximal end portion, a distal end portion having a distal tip, and a wall circumferentially enclosing a sheath lumen extending along an entire length of the sheath;
wherein the delivery catheter is sized to be received and selectively advanceable within the sheath lumen such that the occlusion device can be passed through the sheath lumen to a position distal of the distal tip.

46. The method of claim 45, wherein the sheath further comprises:
at least one inflation channel within the wall of the sheath; and
a balloon coupled to the distal end portion of the sheath and positioned in fluid communication with the at least one inflation channel of the sheath, the balloon enclosing an interior space.

47. The method of claim 46, wherein the wall of the delivery catheter body defines at least one outlet opening to provide fluid communication between the at least one inflation channel and the interior space of the balloon.

48. The method of any of claims 44-47, wherein the at least one injection channel of the delivery catheter body comprises a plurality of injection channels.

49. The method of any of claims 44-48, wherein the delivery catheter further comprises an element configured to irradiate the photocurable biomaterial with actinic radiation, such as a water light pipe, light source (e.g., LED), or a combination thereof.
